(19) United States
(12) Patent Application Publication
(10) Pub. No.: US 2011/0152176 A1
(43) Pub. Date: Jun. 23, 2011

(54) AGR-MEDIATED INHIBITION AND DISPERSAL OF BIOFILMS

(75) Inventor: Alexander R. Horswill, Coralville, IA (US)

(73) Assignee: UNIVERSITY OF IOWA RESEARCH FOUNDATION

(21) Appl. No.: 12/999,038

(22) PCT Filed: May 27, 2009
(86) PCT No.: PCT/US09/45313

§ 371 Date: Mar. 7, 2011

Related U.S. Application Data
(60) Provisional application No. 61/073,175, filed on Jun. 17, 2008.

(51) Int. Cl. A61K 38/16; A61P 31/04

(52) U.S. Cl. 514/2.7; 514/2.8

(57) ABSTRACT

The present invention involves the use of activators of bacterial agr quoroum-sensing systems to block, inhibit or reverse biofilm formation. The biofilm may be located on an industrial or medical surface, or may be located in a subject, such as in a wound or infected organ, or on an in-dwelling medical device.

The present application claims benefit of priority to U.S. Provisional Appln. Ser. No. 61/073,175, filed Jun. 17, 2008, the entire contents of which are incorporated by reference.

### BACKGROUND OF THE INVENTION

1. Field of the Invention

The invention generally concerns methods and compositions for the inhibition of biofilms. More specifically, the invention addresses the use of agr agonists to block or reverse biofilm formation and/or restore sensitivity to antibiotics.

2. Description of Related Art

Most bacteria have an inherent ability to form surface-attached communities of cells called biofilms (Davey & O'Toole, 2000). The opportunistic pathogen Staphylococcus aureus can form biofilms on many host tissues and implanted medical devices often causing chronic infections (Furukawa et al., 2006; Parsek & Singh, 2003; Harris & Richards, 2006; Costerton, 2005). The challenge presented by biofilm infections is the remarkable resistance to both host immune responses and available chemotherapies (Patel, 2005; Leid et al., 2002), and estimates suggest that up to 80% of chronic bacterial infections are biofilm associated (Davies, 2003). In response to certain environmental cues, bacteria living in biofilms can use active mechanisms to leave biofilms and return to the planktonic (free-living) state in which sensitivity to antimicrobials is regained (Fux et al., 2004; Boles et al., 2005; Hall-Stoodley & Stoodley, 2005).

“Quorum-sensing” is a type of decision-making process used by decentralized groups to coordinate behavior. Many species of bacteria use quorum-sensing to coordinate their gene expression according to the local density of their population. Studies on the opportunistic pathogen Pseudomonas aeruginosa indicate that quorum-sensing is required to make a robust biofilm under some growth conditions (Davies et al., 2003). Surprisingly, the opposite is true in S. aureus, as the presence of an active quorum-sensing impedes attachment and development of a biofilm (Vuong et al., 2000; Beenken et al., 2003), with one study by Yarwood et al. (2004) showing that bacteria dispersing from biofilms displayed high levels of agr activity, while cells in a biofilm had predominantly repressed agr systems. Thus, more information is required to fully understand the molecular mechanisms underlying biofilm formation and detachment, and the implications of altering agr quorum sensing.

### SUMMARY OF THE INVENTION

Thus, in accordance with the present invention, there is provided a method of inhibiting a bacterial biofilm comprising contacting a biofilm-forming bacterium with an activator of an agr quorum-sensing system. The agr quorum-sensing system may be agr-I, agr-II, agr-III or agr-IV. The activator may be an auto-inducing peptide (AIP). The bacterium may be Staphylococcus aureus or Psuedomonas aeruginosa. The method may further comprise contacting said bacterium with an antibiotic or antiseptic agent. Inhibiting may comprise inhibiting biofilm formation, inhibiting biofilm growth, reducing biofilm size or promoting detachment of bacteria from a formed biofilm.

The biofilm or biofilm-forming bacterium may be located in a subject, such as a mammalian subject, including a human subject. The subject may comprises an in-dwelling medical device, such as a catheter, a pump, endotracheal tube, a nephrostomy tube, a stent, an orthopedic device, a suture, a or prosthetic valve. The catheter may be a vascular catheter, an urinary catheter, a peritoneal catheter, an epidural catheter, a central nervous system catheter, central venous catheter, an arterial line catheter, a pulmonary artery catheter, or a peripheral venous catheter. The method may thus further comprise coating the in-dwelling medical device with said inhibitor prior to implantation. The biofilm or biofilm-forming bacterium may be located on a wound dressing, or on a tissue surface, such as a heart valve, bone or epithelia.

Alternatively, the biofilm or biofilm-forming bacterium may be located on an inanimate surface, such as a floor, a table-top, a counter-top, a medical device surface, a wheelchair surface, a bed surface, a sink, a toilet, a filter, a valve, a coupling, or a tank. The biofilm may also be located in an industrial system, such as a heating/cooling system, a water provision or purification system, or a medical pump system.

In another embodiment, there is provided a method of preventing a biofilm formation secondary to nosocomial infection in a subject comprising administering to said subject an activator of an agr quorum-sensing system in combination with an antibiotic. The nosocomial infection is pneumonia, bacteremia, a urinary tract infection, a catheter-exit site infection, and a surgical wound infection.

In still another embodiment, there is provided a method of restoring antibiotic sensitivity to a bacterium located in a biofilm comprising contacting said bacterium with an activator of an agr quorum-sensing system. The method may further comprise administerting an antibiotic to said subject.

As used herein the specification, “a” or “an” may mean one or more. As used herein in the claim(s), when used in conjunction with the word “comprising,” the words “a” or “an” may mean one or more than one. The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more. Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings are part of the present specification and are included to further demonstrate certain aspects of the present invention. The invention may be better understood by reference to the drawings in combination with the detailed description of specific embodiments presented herein.

FIGS. 1A-E—Low agr activity is important for S. aureus biofilm formation. Biofilms were grown for 2 days in either 2% TSB or 2% TSB supplemented with 0.2% glucose (referred to as “TSBg”). Biofilm integrity and RFP fluorescence were monitored with CLSM. Three dimensional image reconstructions of a z series were created with Velocity software. CLSM images are representative of three separate experiments and each side of a grid square represents 20 mM. (FIG. 1A) AH596 (agr+) grown in TSB. (FIG. 1B) AH596 grown in TSBg. (FIG. 1C) AH871 (agr−) grown in TSB. (FIG. 1D) AH871 grown in TSBg. (FIG. 1E) Measurement of the agr P3-GFP reporter (pDB59) activity in strains AH596 and AH871 grown in broth culture in either TSB or TSBg. Error bars show standard error of the mean (SEM).

FIGS. 2A-C—Detachment of S. aureus biofilms with AIP. Biofilms (strain AH500) were grown in flow cells for 2 days. Either (FIG. 2A) 1 mL of buffer (100 mM phosphate [pH 7], 50 mM NaCl, 1 mM TCEP) or (FIG. 2B) 1 mL of 20 mM AIP-I in buffer was diluted 1000-fold into the biofilm growth media. The biofilm integrity was monitored with CLSM for 2 more days. Each side of a grid square in the image reconstructions represents 20 mM. (FIG. 2C) Effect of AIP-I addition on number of detached bacteria in the effluent medium from flow cell biofilms. The plot depicts CFU/ml in effluents from biofilms, and the black squares represent AlP-I addition and the black circles represent buffer addition to the biofilm. Graph shows the mean of 3 effluent collections from 1 experiment, error bars show SEM.

FIGS. 3A-C—Effect of AIP addition to biofilms from S. aureus strains representing different agr classes. Biofilms were grown in flow cells for 2 days and indicated AIP was added (50 nM final concentration) to the growth media. Biofilm integrity was monitored with CLSM. Each side of a grid square in the image reconstructions represents 20 mM, and red color is from propidium iodide stain present in growth medium. (FIG. 3A) Biofilm of strain FRI1169 (agr Type I) treated with ALP-I. (FIG. 3B) Biofilm of strain SA502A (agr Type II) treated with AIP-II. (FIG. 3C) Biofilm of strain ATCC25923 (agr Type III) treated with AIP-III.

FIGS. 4A-B—Effect of changing growth conditions on agr-mediated biofilm detachment. Dual-labeled biofilms (PsarA-RFP, PagrP3-GFP) of (FIG. 4A) agr positive strain AH596 and (FIG. 4B) agr mutant strain AH871 were grown for 2 days in TSBg. Glucose was removed from the growth media and the biofilm was grown an additional 2 days. Biofilm integrity and RFP/GFP fluorescence were monitored with CLSM. CLSM image reconstructions are representative of three separate experiments and each side of a grid square represents 20 mM.

FIGS. 5A-C—Expression of agr P3 promoter in biofilms after AIP addition. Dual-labeled biofilms (PsarA-RFP, PagrP3-GFP) were grown for 2 days, and AIP-I (50 nM final) was added to the growth media. Biofilm integrity and RFP/GFP fluorescence were monitored with CLSM at day 3 and 4. Greenish yellow color indicates expression of the agr P3-GFP reporter (pDB59). (FIG. 5A) Addition of AIP-I to an agr type I wild type strain (AH596) or (FIG. 5B) agr deficient strain (AH861). (FIG. 5C) Addition of interfering AIP-II to an agr type-I strain biofilm (AH596). CLSM image reconstructions are representative of three separate experiments and each side of a grid square represents 20 mM.

FIG. 6—Susceptibility of biofilm and detached bacteria to rifampicin killing. S. aureus SH1000 biofilm bacteria (black diamonds) were grown in flow cells containing removable coupons, allowing multiple replicate biofilms to be exposed to rifampicin and surviving CFU's to be determined. Detached bacteria (black circles) were collected from flow cell effluents of biofilms exposed to AIP-I. As a control, planktonic bacteria (black squares) were treated with the same level of rifampicin. Graph show the mean of three experiments; error bars show SEM.

FIGS. 7A-C—Role of ica locus in biofilm development. (FIG. 7A) Microtiter biofilms of ica-positive strain SH1000 and ica deletion mutant AH595. (FIG. 7B) Quantitation of microtiter biofilms. (FIG. 7C) Representative CLSM image of flow cells biofilms of strain AH595 grow in TSBg for 2 days. Each side of a grid square represents 20 mM, and red color is from propidium iodide stain present in growth medium.

FIGS. 8A-C—Effect of Proteinase K on biofilms and measurement of extracellular protease activity in AIP-detached biofilms. (FIG. 8A) Proteinase K (proK, 2 mg/ml) was added to a 2 day old biofilm (strain AHSO0) and the biofilm integrity was monitored with CLSM at 6 and 12 hr. (FIG. 8B) Levels of protease activity detected in biofilm effluent collected from wild-type (SH1000) or Δagr (SH1001) biofilms supplemented with indicated AIP's. Protease activity was referenced to wild-type without AIP addition. (FIG. 8C) The effect of inhibitors or activators on the proteolytic activity in an AIP-I detached biofilm effluent. Activity assay was supplemented with either the metalloprotease inhibitor EGTA (1 mM), serine protease inhibitor PMSF (10 mM), or the reducing agent DTT (1 mM). Error bars show SEM.

FIGS. 9A-D—Effect of a serine protease inhibitor and protease-deficient mutants on AIP-I mediated biofilm detachment. Columns show CLSM reconstructions of biofilms at day 2, day 3 and day 4. Biofilms were grown for 2 days and the growth media was supplemented with AIP-I or AIP-I+PMSF as indicated. Greenish-yellow color indicates expression of the agr P3-GFP reporter, and the red color is from propidium iodide present in the growth medium. (FIG. 9A) Wild-type biofilm (AH462) supplemented with 50 nM AIP-I. (FIG. 9B) Wild-type biofilm (AH462) supplemented with 50 nM AIP-I and 10 mM PMSF. (FIG. 9C) Aureolysin (Δaur) mutant biofilm (AH789) supplemented with 50 nM AIP-I. (FIG. 9D) Aureolysin Sp1 (Δaur Dsp1) double mutant biofilm (AH788) supplemented with 50 nM AIP-I. CSLM reconstructions are representative of three separate experiments and each side of a grid square represents 20 mM. Percent biomass detached was calculated by COMSTAT analysis comparing biomass at day 2 to biomass at day 4.

FIGS. 10A-C—Extracellular protease activity and biofilm formation of protease mutants. (FIG. 10A) Relative protease levels detected in wild-type and protease mutants grown in broth culture. Images show bacterial colonies and zones of clearing caused by protease activity on milk agar plates. (FIGS. 10B-C) Biofilm formation of wild type and protease mutants in wells of microtiter plates. Graphs show quantitation of biofilm biomass attached to microtiter plate grown in either (FIG. 10B) TSBg or (FIG. 10C) TSB. Images below each graph are of crystal violet stained biofilms in wells of microtiter plates.

### DETAILED DESCRIPTION OF THE INVENTION

The majority of studies on biofilm detachment have focused on factors capable of initiating the process, such as nutrient availability (Hunt et al., 2004; Sauer et al., 2004), nitric oxide exposure (Barraud et al., 2006), oxygen tension (Thormann et al., 2005), iron salts (Musk et al., 2005), chelators (Banin et al., 2006), and signaling molecules (Morgan et al., 2006; Rice et al., 2005; Dow et al., 2003; Thormann et al., 2006). Alternatively, detachment studies have addressed effector gene products that contribute to the dissolution of the biofilm, including surfactants (Boles et al., 2005; Vuong et al., 2000; Irie et al., 2005; Davey et al., 2003), hydrolases (Kaplan et al., 2004; Kaplan et al., 2003), proteases (Chaignon et al., 2007; O'Neill et al., 2007; Rohde et al., 2007), and DNase (Whitchurch et al., 2002). Here, the inventors have done both, by demonstrating that the increasing AIP levels or lowering available glucose can function as a S. aureus biofilm detachment signal by activating the agr quorum-sensing system, resulting in increased levels of extracellular proteases needed for the detachment mechanism. Importantly, agr-mediated detachment also restores antibiotic sensitivity to the released bacteria, suggesting the mechanism could be a target for treating biofilm infections.

These results are in accord with previous studies showing that agr mutants have a propensity to form biofilms (Vuong et al., 2000; Beenken et al., 2003) and that cells actively expressing agr leave biofilms at a high frequency (Yarwood et al., 2004). These findings also explain why S. aureus biofilm formation requires glucose supplementation to growth media. Unless the agr system is repressed or inactivated, or the enzymes mediating detachment are inhibited, S. aureus will remain in a planktonic state. The presence of glucose is known to represses RNAIII through a non-maintained pH decrease to about 5.5 (Regassa et al., 1992), resulting from the secretion of acidic metabolites. The RNAIII repression is not due to glucose itself, but results from the mild acid conditions (Weinrick et al., 2004) and can be mimicked with other carbon sources, such as galactose (Regassa et al., 1992), that also lower the media pH. In microtiter biofilm experiments, the inventors found these alternative pH-lowering carbon sources could substitute for glucose in facilitating biofilm formation (data not shown). The molecular mechanism through which low pH inhibits RNAIII expression remains to be determined. In the host, many niches colonized by S. aureus are maintained in lower pH ranges, such as the skin and vaginal tract (Weinrick et al., 2004), colonization sites that repress agr function could promote biofilm formation.

Based on the findings, inventors propose that the S. aureus agr quorum-sensing system controls the switch between planktonic and biofilm lifestyles. When the agr system is repressed, cells have a propensity to attach to surfaces and form biofilms as detachment factors are produced at low levels. In the inventors' detachment model, dispersal of cells from an established biofilm requires reactivation of the agr system and occurs through a protease-mediated, ica-independent mechanism. Yarwood et al. (2004) demonstrated through time-course, flow cell studies that reactivation of agr does occur in a biofilm, presumably through autonomous AIP production that reaches local concentrations high enough to activate agr. Under these fixed conditions, the agr system may function primarily as a mechanism to detach clumps (also called emboli) that seed new colonization sites.

In the experiments presented herein, the inventors have employed growth conditions that tip the balance of the agr system, allowing an investigation into full agr reactivation within an established biofilm. This delicate balance can be offset with an increase in local AIP concentration or through changing environmental conditions, both situations that induce agr and result in massive dispersion of the cells. Biofilms are dynamic and dispersal is always operating (Hall-Stoodley & Stoodley, 2005), but accelerated detachment has been observed in response to changing environmental conditions, such as oxygen levels (Thormann et al., 2005; Applegate and Bryers, 1991), nutrient depletion (Hunt et al., 2004), changing nutrient composition (Sauer et al., 2004), or increased concentration of quorum-sensing signals (Rice et al., 2005). An S. aureus biofilm growing in vivo is likely to encounter a changing physiochemical environment, which could serve as a cue to induce accelerated detachment through an agr-mediated mechanism.

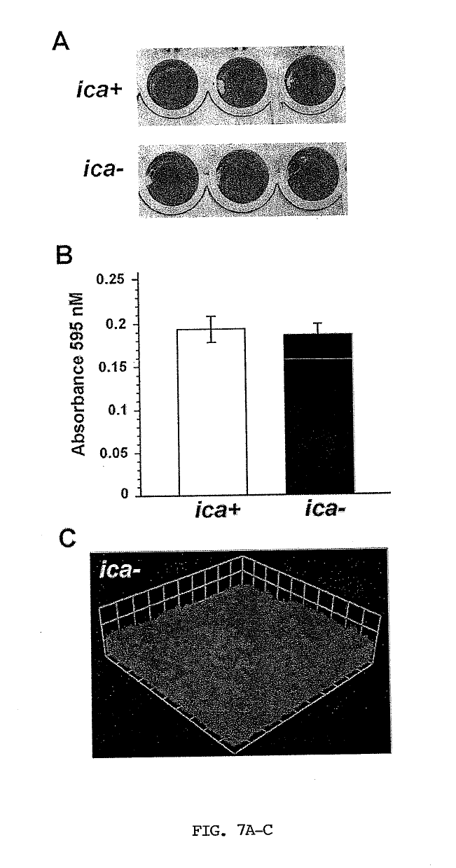

S. aureus has been reported to form biofilms through an ica-dependent mechanism suggesting that PIA could have a role in detachment (O'Gara, 2007; Cramton et al., 1999). The inventors observed no defect in microtiter or flow cell biofilm formation using an ica mutant of SH1000 (FIG. 7). These findings support the growing evidence that PIA is not a major matrix component of S. aureus biofilms, as exogenous addition of dispersin B, an N-acetyl-glucosaminidase capable of degrading PIA, has little effect on established biofilms of SH1000 and other S. aureus strains (Izano et al., 2008). In contrast, dispersin B does detach S. epidermidis biofilms indicating a more significant role for PIA in the S. epidermidis matrix structure (Izano et al., 2008). The inventors' experiments with proteinase K and the S. aureus proteases indicate that some proteinaceous material is important for SH1000 biofilmintegrity, and this result supports a number of recent studies demonstrating that proteases can inhibit biofilm formation or detach established biofilms from many S. aureus strains ((Toledo-Arana et al., 2005; Chaignon et al., 2007; O'Neill et al., 2007; Rohde et al., 2007). It is not clear whether agr-mediated detachment will function in S. aureus strains that produce an ica-dependent biofilm.

In this study, the inventors document a role for the Aur and Sp1 proteases in biofilm detachment. Global expression analysis has shown that activation of the agr quorum-sensing system results in upregulation of extracellular proteases (Aur, Sp1ABCDEF, ScpA, SspAB) and down-regulation of many surface proteins (Dunman et al., 2001; Ziebandt et al., 2004). However, the target of these agr controlled proteases is not clear. One potential target is the surface adhesins, and possible candidates include the surface proteins Atl, Bap, and SasG, all of which have reported roles in biofilm formation (Corrigan et al., 2007; Trotonda et al., 2005; Curcarella et al., 2001; Biswas et al., 2006; Heilmann et al., 1997). Atl is additionally known to require proteolytic processing for activation, and this processing is PMSF inhibited (Oshida et al., 1995). Other possibilities include microbial surface components recognizing adhesive matrix molecules (MSCRAMMs), which are important for adherence to the extracellular matrices of mammalian cells (Clarke and Foster, 2006). Also, the S. aureus secreted proteases are known to activate lipase (Sal-1 and Sal-2) precursors (Gotz et al., 1998) and process other secreted enzymes, such as staphylococcal nuclease (Suciu and Inouye, 1996; Davis et al., 1977).

In addition to proteases, there may be other agr regulated factors that contribute to biofilm detachment. Surfactant-like molecules, such as d-toxin, are induced by the agr system and may exert dispersal effects on biofilms (Vuong et al., 2000; Kong et al., 2006). There is growing evidence that extracellular DNA (eDNA) is an important S. aureus biofilm matrix component (Rice et al., 2007; Izano et al., 2008), and expression of staphylococcal nuclease is reported to be under control of the agr system (Novick, 2003). Thus, while agr-induced proteases are required for the detachment phenotype, the agr-controlled expression of an array of factors (proteases, nuclease, surfactants) may also contribute to the biofilm detachment mechanism.

There is increasing interest in understanding how bacteria detach from biofilms and initiate colonization of new surfaces. The regulation of quorum-sensing systems may be one mechanism by which many bacteria control biofilm formation and dispersal. Quorum-sensing has been implicated in dispersal of biofilms formed by Yersinia pseudotuberculosis (Atkinson et al., 1999), Rhodobacter sphaeroides (Puskas et al., 1997), Pseudomonas aureofaciens (Zhang and Pierson, 2001), Xanthomonas capmestris (Dow et al., 2003), and Serratia marceascens (Rice et al., 2005). However, homoserine lactone signals play a divergent role in Pseudomonas aeuruginosa (Davies et al., 1998), Pseudomonas fluorescens (Allison et al., 1998), and Burkholderia cepacia (Huber et al., 2001), where the active versions of these quorum-sensing system are necessary for biofilm formation and robustness under some growth conditions. In both cases, it appears quorum-sensing plays a significant role in biofilm development and determining the environmental stimuli that modulate quorum-sensing activity will provide insight on bacterial colonization, detachment, and dispersal to new sites.

Thus, the inventors have now demonstrated that activation of the agr system in established biofilms is necessary for detachment. This activation could be accomplished with exogenous AIP addition or by changing nutrient availability to the biofilm. They also demonstrate that agr-mediated detachment requires the activity of extracellular proteases. Together, these findings suggest that agr quorum-sensing is an important regulatory switch between planktonic and biofilm lifestyles that may contribute to S. aureus dispersal and colonization of new sites. It also provides a new target for control of biofilm formation in industrial and therapeutic settings.

### I. AGR QUORUM-SENSING SYSTEMS

A. Quorum-Sensing

Quorum-sensing is a type of decision-making process used by decentralized groups to coordinate behavior. Many species of bacteria use quorum-sensing to coordinate their gene expression according to the local density of their population. Similarly, some social insects use quorum sensing to make collective decisions about where to nest. In addition to its function in biological systems, quorum sensing has several useful applications for computing and robotics. Quorum sensing can function as a decision-making process in any decentralized system, as long as individual components have (a) a means of assessing the number of other components they interact with and (b) a standard response once a threshold number of components is detected.

Some of the best-known examples of quorum-sensing come from studies of bacteria. Bacteria use quorum-sensing to coordinate certain behaviors based on the local density of the bacterial population. Quorum-sensing can occur within a single bacterial species as well as between diverse species, and can regulate a host of different processes, essentially serving as a simple communication network. A variety of different molecules can be used as signals.

Three-dimensional structures of proteins involved in quorum-sensing were first published in 2001, when the crystal structures of three LuxS orthologs were determined by X-ray crystallography. In 2002, the crystal structure of the receptor LuxP of Vibrio harveyi with its inducer AI-2 (which is one of the few biomolecules containing boron) bound to it was also determined. AI-2 signalling is conserved among many bacterial species, including E. coli, an enteric bacterium and model organism for Gram-negative bacteria. Although this conservation has suggested that autoinducer-2 could be used for widespread interspecies communication, a comparative genomic and phylogenetic analysis of 138 genomes of bacteria, archaea, and eukaryotes did not support the concept of a multispecies signaling system relying on AI-2 outside Vibrio species.

The S. aureus quorum-sensing system is encoded by the accessory gene regulator (agr) locus and the communication molecule that it produces and senses is called an autoinducing peptide (AIP), which is an eight-residue peptide with the last five residues constrained in a cyclic thiolactone ring (Ji et al., 1997) mechanism that requires multiple peptidases (Kavanaugh et al., 2007; Qiu et al., 2005). Once AIP reaches a critical concentration, it binds to a surface histidine kinase receptor, initiating a regulatory cascade that controls expression of a myriad of virulence factors, such as proteases, hemolysins, and toxins (Novick, 2003). A recent study by Yarwood et al. (2004) raised the possibility that the agr quorum-sensing system is involved in biofilm detachment. That study demonstrated that bacteria dispersing from biofilms displayed high levels of agr activity, while cells in a biofilm had predominantly repressed agr systems. These findings correlate well with prior data indicating that agr-deficient S. aureus strains form more robust biofilms compared to wild-type strains (Vuong et al., 2000; Beenken et al., 2003). However, the effects of agr modulation of biofilm formation and maintenance have yet to be explored.

B. Bacteria

As discussed above, bacteria that use quorum sensing constantly produce and secrete certain signaling molecules (called autoinducers or pheromones), These bacteria also have a receptor that can specifically detect the signaling molecule (inducer). When the inducer binds the receptor, it activates transcription of certain genes, including those for inducer synthesis. There is a low likelihood of a bacterium detecting its own secreted AHL. Thus, in order for gene transcription to be activated, the cell must encounter signaling molecules secreted by other cells in its environment. When only a few other bacteria of the same kind are in the vicinity, diffusion reduces the concentration of the inducer in the surrounding medium to almost zero, so the bacteria produce little inducer. However, as the population grows the concentration of the inducer passes a threshold, causing more inducer to be synthesized. This forms a positive feedback loop, and the receptor becomes fully activated. Activation of the receptor induces the up regulation of other specific genes, causing all of the cells to begin transcription at approximately the same time.

Vibrio fischeri. Quorum sensing was first observed in Vibrio fischeri, a bioluminiscent bacterium that lives as a mutualistic symbiont in the photophore (or light-producing organ) of the Hawaiian bobtail squid. When V. fischeri cells are free-living (or planktonic), the autoinducer is at low concentration and thus cells do not luminesce. However, when they are highly concentrated in the photophore (about 10¹¹ cells/ml) transcription of luciferase is induced, leading to bioluminescence.

Escherichia coli. In the Gram-negative bacteria Escherichia coli, cell division may be partially regulated by AI-2-mediated quorum sensing. This species uses AI-2, which is produced and processed by the lsr operon. Part of it encodes an ABC transporter which imports AI-2 into the cells during the early stationary (latent) phase of growth. AI-2 is then phosphorylated by the LsrK kinase, and the newly produced phospho-AI-2 can either be internalized or used to suppress LsrR, a repressor of the lsr operon (thereby activating the operon). Transcription of the lsr operon is also thought to be inhibited by dihydroxyacetone phosphate (DHAP) through its competitive binding to LsrR. Glyceraldehyde 3-phosphate has also been shown to inhibit the lsr operon through cAMP-CAPK-mediated inhibition. This explains why when grown with glucose E. coli will lose the ability to internalize AI-2 (because of catabolite repression). When grown normally, AI-2 presence is transient.

Pseudomonas aeruginosa. The opportunistic bacteria Pseudomonas aeruginosa uses quorum sensing to coordinate the formation of biofilms, swarming motility, exopolysaccharide production, and cell aggregation. These bacteria can grow within a host without harming it, until they reach a certain concentration. Then they become aggressive, their numbers sufficient to overcome the host's immune system and form a biofilm, leading to disease. In this species, AI-2 was found to increase expression of sdiA, a transcriptional regulator of promoters which promote ftsQ, part of the ftsQAZ operon essential for cell division. Another form of gene regulation which allows the bacteria to rapidly adapt to surrounding changes is through environmental signaling. Recent studies have discovered that anaerobiosis can significantly impact the major regulatory circuit of QS. This important link between QS and anaerobiosis has a significant impact on production of virulence factors of this organism. It is hoped that the therapeutic enzymatic degradation of the signaling molecules will prevent the formation of such biofilms and possibly weaken established biofilms. Disrupting the signalling process in this way is called quorum quenching.

Staphylococcus aureus. S. aureus controls the expression of extracellular virulence factors through an agr quorum-sensing mechanism. This regulatory cascade responds to the extracellular presence of a secreted peptide signal, also called an autoinducing peptide or AIP. The AIP signals are 7-9 amino acids in length and have the C-terminal five residues constrained as a thiolactone ring through a cysteine side chain. The genes required for the quorum-sensing system are located in the agr locus, a chromosomal region that contains two divergent transcripts, called RNAII and RNAIII. The RNAII transcript encodes the majority of proteins necessary to generate and sense extracellular AIPs, while the RNAIII transcript is a regulatory RNA and the primary effector of the agr system. Like other quorum-sensing molecules, AIPs are produced during growth and accumulate outside the cell until they reach a critical concentration, activating the agr system. The regulatory cascade increases levels of the RNAII and RNAIII transcripts, leading to induction of virulence factor expression (Novick, 2003).

C. Activators of Quorum Sensing Systems

i. AIP Compositions

In certain embodiments, the present invention concerns compositions comprising so-called “auto-inducing peptides” that are involved in quorum-sensing in bacteria. An interesting feature of the S. aureus agr system is the variation among strains (Novick, 2003). There are four different classes of Agr systems each recognizing a unique AIP structure (referred to as Agr-I, Agr-II, Agr-III, and Agr-IV; similarly, their cognate signals are termed AIP-I through AIP-IV). Through a fascinating mechanism of chemical communication, these different AIP signals cross-inhibit the activity of the others with surprising potency, presumably giving a competitive advantage to the producing S. aureus strain. Indeed, Agr interference has been observed with in vivo competition experiments (Fleming et al., 2006), and the addition of an inhibitory AIP will block development of an acute infection (Wright et al., 2005).

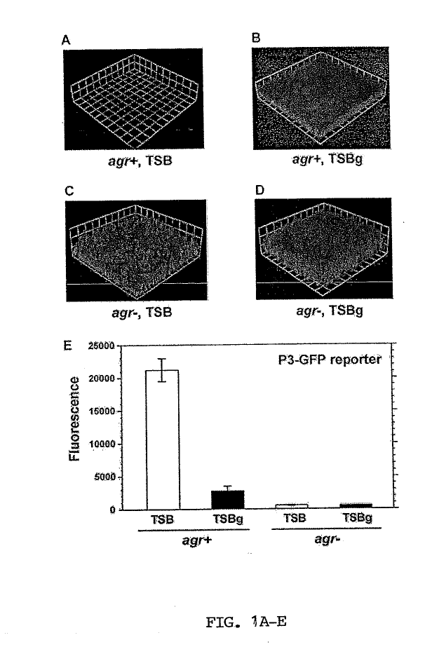

Among the four AIP classes, the five-residue thiolactone ring structure is always conserved, while the other ring and tail residues differ (Malone et al., 2007). Similarly, the proteins involved in signal biosynthesis and surface receptor binding also show variability (Wright et al., 2004; Zhang and Ji, 2004). In Agr interference, there are three classes of cross-inhibitory groups: AIP-I/IV, AIP-II, and AIP-III (FIG. 1). Since AIP-I and AIP-IV differ by only one amino acid and function interchangeably (Jarraud et al., 2000), they are grouped together. The three AIP groups all cross-inhibit each other with binding constants in the low nanomolar range (Lyon et al., 2002; Mayville et al., 1999). Interestingly, the typing of the four Agr systems roughly correlates with specific classes of diseases (Jarraud et al., 2000; Jarraud et al., 2002), although the significance of this observation is unclear.

Studies that have relied on extracellular addition of AIPs have required chemical synthesis of the signal (Sung et al., 2006; Wright et al., 2005). While the strategy has been effective, it is prohibitive for many laboratories, impeding research on the AIP molecules. The AIPs can be purified from culture supernatants (Ji et al., 1997), but the yields are low and the procedures are labor-intensive, making this approach unattractive. The inventor also has reported on a convenient, enzymatic approach to generating AIP molecules (Malone et al., 2007) employing an engineered DnaB mini-intein from Synechocystis sp. strain PCC6803. The sequences of AIP-I to -IV are shown below:

| | | |
|---|---|---|
| AIP-I | YSTCDFIM | SEQ ID NO: 1 |
| AIP-II | GVNACSSLF | SEQ ID NO: 2 |
| AIP-III | INCDFLL | SEQ ID NO: 3 |
| AIP-IV | YSTCYFIM | SEQ ID NO: 4 |

For each peptide, a thiolactone bridge is formed between the C-terminal residues and the underlined internal cysteine reside. Methods of making such peptides are disclosed in PCT US2007/087663, incorporated herein by reference. Other related compounds are described in U.S. Pat. Nos. 6,953,833 and 6,337,385, and U.S. Patent Publication 2007/0185016, incorporated herein by reference.

In certain embodiments, the AIP composition is provided in a biocompatible form. As used herein, the term “biocompatible” refers to a substance which produces no significant untoward effects when applied to, or administered to, a given organism according to the methods and amounts described herein. Such untoward or undesirable effects are those such as significant toxicity or adverse immunological reactions. In particular embodiments, biocompatible protein, polypeptide or peptide containing compositions will generally be proteins or peptides or synthetic proteins or peptides each essentially free from toxins, pathogens and harmful immunogens.

In certain embodiments and as described supra, AIP's may be purified. Generally, “purified” will refer to a protein, polypeptide, or peptide composition that has been subjected to fractionation to remove various other proteins, polypeptides, or peptides, and which composition substantially retains its activity, as may be assessed, for example, by the protein assays, as would be known to one of ordinary skill in the art for the specific or desired protein, polypeptide or peptide.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide or polypeptide are filtration, ion-exchange chromatography, exclusion chromatography, polyacrylamide gel electrophoresis, affinity chromatography, or isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulfate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

ii. Other Activators

Another class of activators for the present invention include inhibitors of the SigB system. One of the most important global regulators in S. aureus, the SigB system is an environmental sensing mechanism used by diverse Gram-positive bacteria to coordinate gene expression. The best characterized of these regulatory networks is from Bacillus subtilis and contains numerous proteins involved in sensing a variety of stresses, including heat, high salt, and alkaline shock conditions (Pane-Farre et al., 2006), and these signals are transmitted them through a cascade to activate SigB. Based on the presence of a SigB gene in other Gram positives, it has long been assumed that the system function is conserved throughout the Gram positives.

However, recent studies in S. aureus demonstrate that the activation mechanism and output regulon shares little resemblance to B. subtilis paradigm (Pane-Farre et al., 2006). In S. aureus, SigB regulates many factors related to virulence, such as carotenoid, hemolysins, extracellular invasive enzymes, polysaccharide intracellular adhesin (PIA), and biofilm formation (Kullik et al., 1997; Horsburgh et al., 2002; Rachid et al., 2000; Bischoff et al., 2004; Ziebandt et al., 2004; 2001; Gertz et al., 1999; Cheung et al., 1999). While many features of the B. subtilis and S. aureus Sigma B systems are different, the Rsb and SigB proteins are similar based on sequence identity. Based on the B. subtilis model, it is assumed that the S. aureus Rsb proteins operate as a protein-protein interaction cascade to modulate SigB activity. Briefly, under environmental stress conditions (heat, base, salt), RsbU dephosphorylates RsbV protein, allowing RsbV and RsbW to interact. With RsbW bound, SigB is free to activate transcription. Under normal growth conditions, RsbV remains phosphorylated, and RsbW functions as an anti-sigma factor and sequesters SigB. While genetic and molecular analysis supports this model, there is little biochemical evidence to verify it. Further, it is not clear how signals are transmitted into the RsbU protein. B. subtilis has a complex sensory component that is completely missing in S. aureus (Pane-Farre et al., 2006). Considering all the S. aureus virulence factors regulated by SigB, it is surprising that these basic features of the system remain unknown.

Similar to animal model studies, the role of SigB in S. aureus biofilm formation has also been controversial. Initial reports on S. aureus SigB defective strains indicated they were unable to form a biofilm (Rachid et al., 2000). However, a later study contradicted these reports and claimed the SigB biofilm phenotype was due to regulation of SarA (Valle et al., 2003), which is known to contain at least one SigB-dependent promoter. In S. epidermidis, it is known that SigB is required to express PIA (Knobloch et al., 2001; 2004), explaining the biofilm defect of SigB mutants in this organism. There has been speculation that SigB regulation of PIA also explains the S. aureus biofilm phenotypes, but growing number of clinical strains produce PIA-independent (ica-independent) biofilms (Izano et al., 2008; O'Neill et al., 2007), especially among the MRSA isolates. Interestingly, overexpression of SigB greatly improves attachment to various human matrices (Entenza et al., 2005). In the inventor's screens for biofilm defective S. aureus mutants, they found multiple insertions in the rsbUVW-sigB locus, and follow-up studies indicate that SigB is important for biofilm formation. Under certain conditions, such as SigB inactivation, high level production of extracellular enzymes ensues and biofilm formation is blocked, and thus the inventor speculates these enhanced exoenzyme levels are the reason for the biofilm phenotypes. Based on these observations, the inventor proposes a model to explain the role of SigB in biofilms. In brief, when an environmental cue induces the SigB system, S. aureus will preferentially form a biofilm, and when SigB is repressed, cells will remain planktonic or leave an established biofilm.

Thus, the present invention contemplates the use of inhibitors of the SigB pathway as a means for activating quorum-sensing in bacteria to prevent biofilms. Such inhibitors may be pharmaceutical “small molecules,” or them may be biologicals, as discussed below.

Antisense Constructs. An alternative approach to inhibiting TRPC is antisense. Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

Ribozymes. Another general class of inhibitors is ribozymes. Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cook et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cook et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). It has also been shown that ribozymes can elicit genetic changes in some cells lines to which they were applied; the altered genes included the oncogenes H-ras, c-fos and genes of HIV. Most of this work involved the modification of a target mRNA, based on a specific mutant codon that was cleaved by a specific ribozyme.

RNAi. RNA interference (also referred to as “RNA-mediated interference” or RNAi) is another mechanism by which TRPC expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin et al., 1999; Montgomery et al., 1998; Sharp et al., 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin et al., 1999; Montgomery et al., 1998; Sharp, 1999; Sharp et al., 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp, 1999; Sharp et al., 2000; Elbashir et al., 2001). It is generally accepted that RNAi acts post-transcriptionally, targeting RNA transcripts for degradation. It appears that both nuclear and cytoplasmic RNA can be targeted (Bosher et al., 2000).

siRNAs must be designed so that they are specific and effective in suppressing the expression of the genes of interest. Methods of selecting the target sequences, i.e. those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis; through processing of longer, double stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single stranded RNA-oligomers followed by the annealing of the two single stranded oligomers into a double stranded RNA. Methods of chemical synthesis are diverse. Non-limiting examples are provided in U.S. Pat. Nos. 5,889,136, 4,415,732, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides +3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically-synthesized siRNAs are found to work optimally when they are in cell culture at concentrations of 25-100 nM. This had been demonstrated by Elbashir et al. (2001) wherein concentrations of about 100 nM achieved effective suppression of expression in mammalian cells. siRNAs have been most effective in mammalian cell culture at about 100 nM. In several instances, however, lower concentrations of chemically synthesized siRNA have been used (Caplen et al., 2000; Elbashir et al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single stranded RNA is enzymatically synthesized from the PCR™ products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

Treatment regimens would vary depending on the clinical situation. However, long term maintenance would appear to be appropriate in most circumstances. It also may be desirable treat hypertrophy with inhibitors of TRP channels intermittently, such as within brief window during disease progression.

Antibodies. In certain aspects of the invention, antibodies may find use as inhibitors or TRPCs. As used herein, the term “antibody” is intended to refer broadly to any appropriate immunologic binding agent such as IgG, IgM, IgA, IgD and IgE. Generally, IgG and/or IgM are preferred because they are the most common antibodies in the physiological situation and because they are most easily made in a laboratory setting.

The teini “antibody” also refers to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art.

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

Single-chain antibodies are described in U.S. Pat. Nos. 4,946,778 and 5,888,773, each of which are hereby incorporated by reference.

“Humanized” antibodies are also contemplated, as are chimeric antibodies from mouse, rat, or other species, bearing human constant and/or variable region domains, bispecific antibodies, recombinant and engineered antibodies and fragments thereof Methods for the development of antibodies that are “custom-tailored” to the patient's dental disease are likewise known and such custom-tailored antibodies are also contemplated.

### II. SCREENING METHODS

The present invention further comprises methods for identifying agents that inhibit the agr quorum-sensing systems of various bacteria. These assays may comprise random screening of large libraries of candidate substances; alternatively, the assays may be used to focus on particular classes or sequences of compounds selected with an eye towards structural attributes that are believed to make them more likely result in a particular biological function, such as antibiotic activity. One example would be mimetics of AIPs, while another would be a SigB-family inhibitor.

To identify a biologically active candidate substance, one generally will determine the a specific biological activity (e.g., cell or biofilm growth, biofilm formation, biofilm detachment) in the presence and absence of the candidate substance. For example, a method generally comprises:
(a) providing a candidate substance;
(b) admixing the candidate polypeptide with a biofilm-forming bacterial cell or biofilm, either in vitro or in a suitable experimental animal;
(c) measuring one or more quorum-sensing characteristics of the cell, biofilm or animal in step (b); and
(d) comparing the characteristic measured in step (c) with the characteristic of the cell, biofilm or animal in the absence of said candidate polypeptide, wherein a difference between the measured characteristics indicates that said candidate modulator is, indeed, a modulator of cell, biofilm or animal.
   It will, of course, be understood that such screening methods are useful in themselves notwithstanding the fact that effective candidates may not be found. The invention provides methods for screening for such candidates, not solely methods of finding them.

A. Modulators

As used herein the term “candidate substance” refers to any molecule that may potentially inhibit or enhance agr quorum sensing. It may prove to be the case that the most useful pharmacological compounds will be compounds that are structurally related to AIP peptides, such as those from S. aureus. Using lead compounds to help develop improved compounds is know as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs that are more active or stable than the natural molecules, which have different susceptibility to alteration or which may affect the function of various other molecules. In one approach, one would generate a three-dimensional structure for a target molecule, or a fragment thereof.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Candidate substances may include fragments or parts of naturally-occurring compounds, or may be found as active combinations of known compounds, which are otherwise inactive. It is proposed that amino acid sequences isolated from natural sources, such as animals, bacteria, fungi, plant sources, may be assayed as candidates for the presence of potentially useful pharmaceutical agents. It will be understood that the pharmaceutical agents to be screened could also be derived or synthesized from chemical compositions or man-made compounds.

In addition to the modulating compounds initially identified, the inventor also contemplates that other sterically similar compounds may be formulated to mimic the key portions of the structure of the modulators.

A biofilm inhibitor according to the present invention may be one which exerts its activating effect upstream, downstream or directly on a a quorum sensing system. Regardless of the type of activator identified by the present screening methods, the effect of the activator by such a compound results in discernable biological changes compared to that observed in the absence of the added candidate substance.

B. In Vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates (e.g., multiwell plates), dishes and other surfaces such as dipsticks or beads.

One example of a cell free assay is a binding assay. While not directly addressing function, the ability of molecule to bind to a target in a specific fashion is strong evidence of a related biological effect. For example, binding of a molecule to a target may, in and of itself, be inhibitory, due to steric, allosteric or charge-charge interactions. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

C. In Cyto Assays

The present invention also contemplates the screening of candidate substances for their ability to modulate quorum sensing pathways in cells. Various cell lines can be utilized for such screening assays, including cells specifically engineered for this purpose. For example, in some aspects, the effect of the candidate substances on cell or biofilm growth may be assessed. In still other cases cells for an in cyto assay may comprise a reporter gene indicating the activity or inhibition of a quorum sensing pathway. For instance, cells may be bacterial cells that express a reporter gene under the control of a promoter that responds to quorum sensing pathways. Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

D. In Vivo Assays

In vivo assays involve the use of various animal models. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more candidate substances are administered to an animal, and the ability of the candidate substance(s) to alter one or more characteristics, as compared to a similar animal not treated with the candidate substance(s), identifies a modulator. The characteristics may be any of those discussed above with regard to the function of a particular compound.

Treatment of these animals with candidate substances will involve the administration of the compound, in an appropriate fowl, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a candidate substance in vivo may involve a variety of different criteria. Also, measuring toxicity and dose response can be performed in animals in a more meaningful fashion than in in vitro or in cyto assays.

### III. METHODS

A. Methods of Treating Subjects

The present invention contemplates, in one embodiment, the treatment of subjects suffering from biofilm formation or at risk of biofilm formation due to various medical or environmental conditions. A variety of medical situations lend themselves to risk of biofilm involvement. For example, patients on chronic antibiotic therapy, immunosuppressed patients, patients having had surgery, and patients with traumatic wounds all are at risk of developing biofilm-type infections.

Administration of pharmaceutical compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions. Upon formulation, solutions will be administered in a manner compatible with the dosage formulation and in such amount as is therapeutically effective.

In one specific embodiment, it is contemplated that the compositions of the present invention will find use in the treatment of S. aureus related-infective endocarditis (Fowler et al., 2005), a complex biofilm (vegetation) of bacteria and host components on a cardiac valve. The pathogenesis of endocarditis initiates with trauma to endothelial layer, followed by formation of sterile clot (thrombus) composed of fibrin and platelets. S. aureus possesses an array of microbial surface components recognizing adhesive matrix molecules (MSCRAMMs) that bind the thrombus, allow microcolony formation, and eventually mature into a dense vegetation (Parsek and Singh, 2003). Numerous complications arise from these biofilms, including congestive heart failure, embolization leading to stroke, mycotic aneurysms, renal dysfunction, and brain abscesses (Bashore et al., 2006). Treatment of valve biofilms is notoriously difficult, with 200-fold higher levels of antibiotics required to eradicate the infection (Joly et al., 1987), often only after weeks of administration (Bashore et al., 2006). Thus, the present invention can be used as a mono- or combination therapy with antibiotics in the treatment of such infections.

B. Pharmaceutical Formulations

Where clinical applications are contemplated, it will be necessary to prepare pharmaceutical compositions—AIPs and other agr quorum-sensing signaling agents—in a form appropriate for the intended application. Generally, this will entail preparing compositions that are essentially free of pyrogens, as well as other impurities that could be harmful to humans or animals.

One will generally desire to employ appropriate salts and buffers to agents stable and allow for uptake by target cells. Aqueous compositions of the present invention comprise an effective amount of the agent to cells or a subject, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the agents of the present invention, its use in therapeutic compositions is contemplated. Supplementary active ingredients also can be incorporated into the compositions.

C. Combination Therapy

Antibiotic resistance represents a major problem in microbiology, and in particular, in the treatment of biofilms. A major goal of current research is to find ways to improve the efficacy of standard antibiotics, and one way is by combining such traditional therapies with a sensitizing or augmenting agent. Thus, in accordance with the present invention, one may kill bacteria, inhibit bacteria or biofilm growth, inhibit biofilm development or spread, induce detachment of a biofilm-involved bacterium or re-establish antibiotic sensitivity of a bacteria or biofilm, one would generally contact a “target” bacterium, biofilm or subject with an agr quorum-sensing agent and at least one other agent. These compositions would be provided in a combined amount effective to achieve any of the foregoing goals. This process may involve contacting the bacteria, biofilm or subject with the agr quorum-sensing agent and the other agent(s) or factor(s) at the same time. This may be achieved by contacting the cell with a single composition or pharmacological formulation that includes both agents, or by contacting the cell with two distinct compositions or formulations, at the same time, wherein one composition includes the agr quorum-sensing agent and the other includes the other agent. Alternatively, the agr quorum-sensing agent therapy treatment may precede or follow the other agent treatment by intervals ranging from minutes to weeks. In embodiments where the other agent and agr quorum-sensing agent are applied separately to the bacteria, biofilm or subject, one would generally ensure that a significant period of time did not expire between the time of each delivery, such that the other agent and agr quorum-sensing agent would still be able to exert an advantageously combined effect on the bacteria, biofilm or subject. In such instances, it is contemplated that one would contact both modalities within about 12-24 hours of each other and, within about 6-12 hours of each other, within about 6 hours of each other, within about 3 hours of each other or within about 1 hour of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of agr quorum-sensing agent or the other agent will be desired. Various combinations may be employed, where agr quorum-sensing agent is “A” and the other agent is “B”, as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated. Antibiotics thay may be employed are include the aminoglycosides (Amikacin (IV), Gentamycin (IV), Kanamycin, Neomycin, Netilmicin, Paromomycin, Streptomycin (IM), Tobramycin (IV)), the carbapenems (Ertapenem (IV/IM), Imipenem (IV), Meropenem (IV)), Chloramphenicol (IV/PO), the fluoroquinolones (Ciprofloxacin (IV/PO), Gatifloxacin (IV/PO), Gemifloxacin (PO), Grepafloxacin* (PO), Levofloxacin (IV/PO), Lomefloxacin, Moxifloxacin (IV/PO), Norfloxacin, Ofloxacin (IV/PO), Sparfloxacin (PO), Trovafloxacin (IV/PO)), the glycopeptides (Vancomycin (IV), the lincosamides (Clindamycin (IV/PO), macrolides/ketolides (Azithromycin (IV/PO), Clarithromycin (PO), Dirithromycin, Erythromycin (IV/PO), Telithromycin), the cephalosporins (Cefadroxil (PO), Cefazolin (IV), Cephalexin (PO), Cephalothin, Cephapirin, Cephradine, Cefaclor (PO), Cefamandole (IV), Cefonicid, Cefotetan (IV), Cefoxitin (IV), Cefprozil (PO), Cefuroxime (IV/PO), Loracarbef (PO), Cefdinir (PO), Cefditoren (PO), Cefixime (PO), Cefoperazone (IV), Cefotaxime (IV), Cefpodoxime (PO), Ceftazidime (IV), Ceftibuten (PO), Ceftizoxime (IV), Ceftriaxone (IV), Cefepime (IV)), monobactams (Aztreonam (IV)), nitroimidazoles (Metronidazole (IV/PO)), oxazolidinones (Linezolid (IV/PO)), penicillins (Amoxicillin (PO), Amoxicillin/Clavulanate (PO), Ampicillin (IV/PO), Ampicillin/Sulbactam (IV), Bacampicillin (PO), Carbenicillin (PO), Cloxacillin, Dicloxacillin, Methicillin, Mezlocillin (IV), Nafcillin (IV), Oxacillin (IV), Penicillin G (IV), Penicillin V (PO), Piperacillin (IV), Piperacillin/Tazobactam (IV), Ticarcillin (IV), Ticarcillin/Clavulanate (IV)), streptogramins (Quinupristin/Dalfopristin (IV), sulfonamide/folate antagonists (Sulfamethoxazole/Trimethoprim (IV/PO)), tetracyclines (Demeclocycline, Doxycycline (IV/PO), Minocycline (IV/PO), Tetracycline (PO)), azole antifungals (Clotrimazole, Fluconazole (IV/PO), Itraconazole (IV/PO), Ketoconazole (PO), Miconazole, Voriconazole (IV/PO)), polyene antifungals (Amphotericin B (IV), Nystatin), echinocandin antifungals (Caspofungin (IV), Micafungin), and other antifungals (Ciclopirox, Flucytosine (PO), Griseofulvin (PO), Terbinafine (PO)).

D. Medical Devices

The invention also provides methods treat or prevent biofilms on medical devices composed of a wide variety of materials. Some examples of those materials include latex, latex silicone, silicone, and polyvinyl chloride. Some examples of devices include endotracheal tubes, vascular catheters, including central venous catheters, arterial lines, pulmonary artery catheters, peripheral venous catheters, urinary catheters, nephrostomy tubes, stents such as biliary stents, peritoneal catheters, epidural catheters, naso-gastric and nasojejunal tubes, central nervous system catheters, including intraventricular shunts and devices, prosthetic valves, and sutures.

In one aspect, the invention comprises pre-treatment of devices prior to implant, thereby effectively reducing or preventing biofilm growth on the device once emplanted. Alternatively, the device may be treated in vivo to prevent, limit, reduce or eliminate biofilms. As discussed above, the agr quorum sensing agonists of the present invention may be used in combinations with antibiotics, and such is contemplated in the medical implant embodiment as well.

E. Industrial Systems

Biofilms may adhere to surfaces, such as pipes and filters and may induce corrosion or fouling of a suface or a manchine. The surface or machine may be comprised in an oil and gas well drilling systems, heating-cooling systems, water filtration systems, such as in swimming pools or water purification plants, countertops, a floors, or food processing tools/equipments. Deleterious biofilms are problematic in industrial settings because they cause fouling and corrosion in systems such as heat exchangers, oil pipelines, and water systems. Biofilms are clearly the direct cause or potentiators for many cooling system problems. Several years ago, the economic impact of biofilms in the U.S. alone was estimated at $60,000,000,000.

Biofilm deposits increase corrosion of metallurgy. The colonization of surfaces by microorganisms and the products associated with microbial metabolic processes create environments that differ greatly from the bulk solution. Low oxygen environments at the biofilm/substrate surface, for example, provide conditions where highly destructive anaerobic organisms such as sulfate reducing bacteria can thrive. This leads to MIC (microbially induced corrosion), a particularly insidious form of corrosion which, according to one published report, can result in localized, pitting corrosion rates 1000-fold higher than that experienced for the rest of the system. In extreme cases, MIC leads to perforations, equipment failure, and expensive reconditioning operations within a short period of time. For example, it has been indicated that in a newly-built university library without an effective microbiological control program sections of the cooling system pipework had to be replaced after just one year of service due to accumulations of sludge, slime, and sulfate-reducing bacteria.

Biofouling may be a biofilm problem which is operationally defined. It applies to biofilms which exceed a given threshold of interference. Biofouling or biological fouling caused by biofilms is the undesirable accumulation of microorganisms on submerged structures, especially ships' hulls. Biofouling is also found in membrane systems, such as membrane bioreactors and reverse osmosis spiral wound membranes. In the same manner, it is found as fouling in cooling water cycles of large industrial equipments and power stations. Anti-fouling is the process of removing the accumulation, or preventing its accumulation.

Biofilm inhibitors can be employed to prevent microorganisms from adhering to surfaces which may be porous, soft, hard, semi-soft, semi-hard, regenerating, or non-regenerating. These surfaces include, but are not limited to, polyurethane, metal, alloy, or polymeric surfaces in medical devices, enamel of teeth, and cellular membranes in animals, preferably, mammals, more preferably, humans. The surfaces may be coated, impregnated or immersed with the biofilm inhibitors prior to use. Alternatively, the surfaces may be treated with biofilm inhibitors to control, reduce, or eradicate the microorganisms adhering to these surfaces.

### IV. EXAMPLES

The following examples are included to further illustrate various aspects of the invention. It should be appreciated by those of skill in the art that the techniques disclosed in the examples that follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Strains and growth conditions. The bacterial strains and plasmids used in this study are described in Table 1. S. aureus or Escherichia coli were grown in tryptic soy broth (TSB) or on tryptic soy agar (TSA) with the appropriate antibiotics for plasmid selection or maintenance (erythromycin 10 mg/ml; chloramphenicol 10 μg/ml; tetracycline 5 μg/ml) and incubated at 37° C. Plasmid DNA was prepared from E. coli and transformed by electroporation into S. aureus RN4220 as described (Schenk and Laddaga, 1992). Plasmids were moved from RN4220 into other S. aureus strains by transduction with bacteriophage a80 (Novick, 1991) or by purifying the plasmid DNA and transformed by electroporation into appropriate strains. To move sspA and sp1ABCDEF mutations into appropriate genetic backgrounds, phage transduction with a80 was used as described (Novick, 1991). To construct the Daur mutation, the pKOR1-aur plasmid was used as described (Kavanaugh et al., 2007). Fluorescence measurements with S. aureus strains containing pDB59 were performed as previously described (Malone et al., 2007).

TABLE 1
Strain and Plasmid List

| Strain or Plasmid | Genotype | Resistance | Source or Reference |
|---|---|---|---|
| Escherichia coli | | | |
| DH5α-E | Cloning strain | None | Invitrogen |
| AH394 | ER2566/ΔgshA::cat | Cam | (Malone et al., 2007) |
| AH426 | AH394/pDnaB8-AIPI | Amp | (Malone et al., 2007) |
| AH495 | AH394/pDnaB8-AIPII | Amp | (Malone et al., 2007) |
| AH496 | AH394/pDnaB8-AIPIII | Amp | (Malone et al., 2007) |
| Staphylococcus | | | |
| aureus | | | |
| RN4220 | restriction mutant of 8325-4 | None | (Novick, 1991) |
| SH1000 | rsbU positive derivative of | None | (Horsburgh et al., 2002) |
| | 8325-4, agr Type I | | |
| SH1001 | SH1000/Δagr::tet | Tet | (Horsburgh et al., 2002) |
| FRI1169 | agr Type I | None | (Sloane et al., 1991) |
| SA502A | agr Type II | None | (Ji et al., 1997) |
| ATCC25923 | agr Type III | None | ATCC |
| KB600 | Δspl::erm | Erm | (Reed et al., 2001) |
| SP6391 | sspA::erm | Erm | (Rice et al., 2001) |
| DU1126 | sspA::tet | Tet | (Blevins et al., 2002) |
| MN8 | Δica::tet | Tet | (Maira-Litran et al., 2002) |
| AH462 | SH1000/pDB59 | Cam | (Kavanaugh et al., 2007) |
| AH500 | SH1000/pAH9 | Erm | This work |
| AH595 | SH1000/Δica::tet | Tet | This work |
| AH596 | SH1000/pD859 + pAH9 | Cam, Erm | This work |
| AH703 | SH1000/Δaur | None | This work |
| AH741 | SH1000/sspA::erm | Erm | This work |
| AH751 | SH1000/Δspl::erm | Erm | This work |
| AH750 | SH1000/Δaur Δspl::erm | Erm | This work |
| AH788 | AH750/pDB59 | Cam, Erm | This work |
| AH789 | AH703/pDB59 | Cam | This work |
| AH860 | SH1000/Δspl::erm | Erm, Tet | This work |
| | sspA::tet | | |
| AH861 | SH1001/pDB59 + pAH9 | Cam, Erm | This work |
| Plasmids | | | |
| pDB59 | P₃-GFP reporter | Amp, Cam | (Yarwood et al., 2004) |
| pAH9 | sarA promoter P₁-RFP | Amp, Erm | This work |
| pDNAB8-AIPI | AIP-I intein plasmid | Amp | (Malone et al., 2007) |
| pDNAB8-AIPII | AIP-II intein plasmid | Amp | (Malone et al., 2007) |
| pDNAB8-AIPIII | AIP-III intein plasmid | Amp | (Malone et al., 2007) |
| pKOR1-aur | aur knockout vector | Amp, Cam | (Kavanaugh et al., 2007) |

Construction of an RFP reporter plasmid. The sarA P1 promoter region was PCR amplified from SH1000 genomic DNA with oligonucleotides (for 5′-TTGTTAAGCTTCTGATATTTTTGACTAAACCAAATGC-3′ (SEQ ID NO:5) rev 5′-TTGGATCCGATGCATCTTGCTCGATACATTTG-3′ (SEQ ID NO:6), digested with HindIII and BamHI, and cloned into the erythromycin shuttle plasmid pCE107 (Yarwood et al., 2004). The mCherry (RFP) gene was PCR amplified from pRSET-mCherry (Shaner et al., 2004) with oligonucleotides incorporating a 5′ ribosome binding site and KpnI site and a 3′ coRI site (for 5′-TTGGTACCTAGGGAGGTTTTAAACATGGTGAGCAAGGGCGAGGAGG-3′ (SEQ ID NO:7) rev 5′-TTGAATTCTTACTTGTACAGCTCGTCCATGCC-3′ (SEQ ID NO:8). The mCherry fragment was cut with KpnI and EcoRI and cloned downstream of the sarA promoter to generate a constitutive RFP expressing plasmid called pAH9.

Monitoring protease activity. Milk agar plates for detection of protease activity consisted of 3 g/L Tryptic Soy broth, 20 g/L non-fat dry milk, and 15 g/L agar. To determine relative protease activities of strains, assays were performed as described previously using the Azocoll (Calbiochem) reagent (Fournier et al., 2001). For measuring protease levels in biofilm effluents, 100 mL of effluent was collected on ice (about 12 hours) after AIP addition to the biofilm medium. Cells were removed from the effluents through centrifugation and filtering, and ammonium sulfate was added to 60% over one hour at 4° C. to concentrate proteins. The precipitated proteins were pelleted by centrifugation at 19,000 rpm for 30 min, and the pellet was washed and resuspended in 1 ml with 10 mM Tris pH 7.5. For the protease assay, the reaction mixture was supplemented with either 1 mM EGTA, 200 mM PMSF, or 1 mM DTT to gauge relative levels of protease classes.

Biofilm experiments. Microtiter plate biofilms were performed as described (Shanks et al., 2005) except that the plates were incubated at 37° C. with shaking at 200 rpm for 12 hours. For flow cell experiments, AIPs were generated using the DnaB intein method, and the AIP concentrations were determined as previously described (Malone et al., 2007). AIPs stocks (20 mM) were stored in 100 mM phosphate (pH 7), 50 mM NaC1, 1 mM tris(2-carboxyethyl) phosphine (TCEP) and were diluted into the biofilm flow medium to a final concentration of 50 nM. When required, 5 mg/ml of erythromycin and/or chloramphenicol were added to the flow cell media to maintain plasmids. The growth medium for flow cell biofilms consisted of 2% TSB plus 0.2% glucose unless otherwise indicated. Flow cell biofilm experiments and confocal microscopy were performed as previously described (Yarwood et al., 2004). Flow cells were inoculated with overnight cultures diluted 1:100 in sterile water and laminar flow (170 ml/min) was initiated after one hour incubation. Confocal microscopy was performed using a Radiance 2100 system (Biorad) with a Nikon Eclipse E600 microscope. Confocal images were processed using Velocity software (Improvision, Lexington, Mass.). Biofilm biomass was quantified with the COMSTAT program (Heydorn et al., 2000) and percent biomass detached was calculated by subtracting biomass present at day 4 from day 2. To quantitate the number of bacteria detaching from a biofilm, 1 ml of flow cell effluent was collected on ice at indicated time points. The collected effluent was vortexed and sonicated in a water bath for 10 minutes to break up clumps, and serial dilutions were plated on TSA plates to determine colony forming units (CFUs). For the Proteinase K detachment experiments, the enzyme (Sigma-Aldrich) was suspended in water and added to the media reservoir at a final concentration of 2 mg/ml.

Antibiotic sensitivity. S. aureus biofilms were grown for two days in a flow chamber lined with removable polycarbonate coupons (Flow Cell FC271, Biosurface Technologies, Bozeman Mont.). Biofilm effluents were collected on ice about 24 hours after AIP-I addition. In parallel, coupons with biofilm growth were removed from flow cells not exposed to AIP-I. Both detached bacteria and the biofilms were exposed to the indicated levels of rifampicin for six hours. Subsequently, cells were vortexed, and the coupons were sonicated in a water bath to break up the biofilm or cell clumps. Serial dilutions were plated on TSA to determine surviving CFU's.

### Example 2

### Results

Low agr activity is important for biofilm development. Mutations in the agr quorum-sensing system are known to improve biofilm development (Vuong et al., 2000; Beenken et al., 2003). Based on these studies, it seemed probable that there is a correlation between agr activity and biofilm formation. Regassa et al. (1992) reported that growth on rich media containing glucose represses the agr system through the non-maintained generation of low pH. Interestingly, in most published flow cell biofilm studies, one commonality is the use of growth media containing or supplemented with glucose (Fux et al., 2004; Yarwood et al., 2004; Rupp et aL, 2005; Beenken et al., 2004; Caiazza and O′Toole, 2003; Rice et al., 2007). In their efforts to grow S. aureus flow cell biofilms, the inventors found a strict dependence on glucose supplementation. For the experimental setup, a once-through, continuous culture system was employed as previously described (Yarwood et al., 2004; Davies et al., 1993), and S. aureus SH1000 constitutively expressing red fluorescent protein (PsarARFP, plasmid pAH9) was used as the testing strain. Using 2% TSB as the growth media, SH1000 cells did not attach and develop a biofilm (FIG. 1A), instead passing right through the flow cell to the effluent. However, in the presence of 0.2% glucose (TSBg), cells attached and a formed a robust biofilm (10-20 microns thick) after two days of growth, which was visually evident and monitored with confocal laser scanning microscopy (CLSM, FIG. 1B). As expected, glucose strongly inhibited expression from the P3 promoter using a GFP reporter (FIG. 1E), suggesting that repression of RNAIII is essential for attachment and biofilm formation. In broth culture and biofilm effluents, the inventors observed a glucose-dependent pH decrease to the 5.5 range similar as previously reported (Regassa et al., 1992; Weinrick et al., 2004). As a control, flow cell biofilms were prepared with an agr mutant strain (SH1001, Dagr::TetM) containing plasmid pAH9 (FIGS. 1C-D), and this strain developed a biofilm even in the absence of media supplementations (FIG. 1C). As anticipated, the P3 promoter did not activate in the agr mutant (FIG. 1E). Overall, these observations indicate that environmental conditions favoring low agr activity are essential for attachment and biofilm formation.

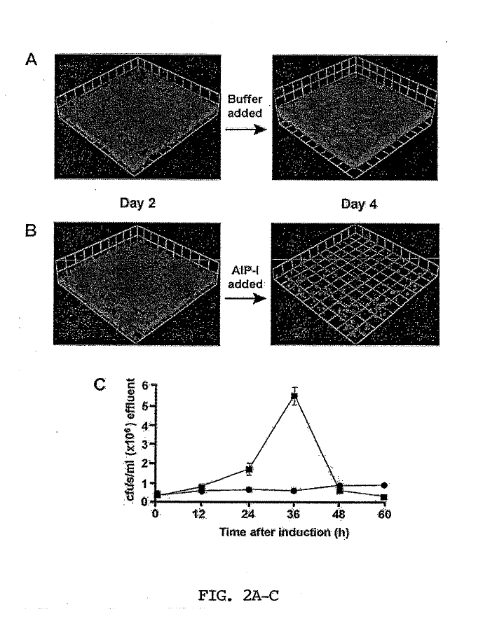

AIP detaches S. aureus biofilms. To investigate the role of the agr system in established biofilms, the inventors developed strategies to modulate level of agr activity within a biofilm. Initially, media supplementation experiments were performed using purified AIP signal in order to place the agr system under external control. The inventors recently developed a new method for AIP biosynthesis (Malone et al., 2007), enabling the production of sufficient signal levels for flow cell experiments. Through exogenous AIP addition, they could test wild-type strains and avoid any potential complications of constructed agr deletion mutants. For this approach, established flow cell biofilms were prepared using S. aureus SH1000 constitutively expressing RFP with plasmid pAH9. The flow cell media was supplemented with glucose to attenuate agr expression (Regassa et al., 1992), allowing cell attachment and biofilmdevelopment. After two days, either 1 mL of buffer (100 mM phosphate [pH 7], 50 mMNaCl, 1 mMTCEP; FIG. 2A) or 1 mL of 20 mMAIP-I in buffer (FIG. 2B and Video S1) was diluted 1000-fold (50 nM final concentration) into the growth media. Using the inventors' synthesized AIP-I in dose-response curves (Malone et al., 2007), the inventors estimate the amount of AIP-I in supernatants of TSB broth cultures (OD600 1.0-1.3) reaches approximately 400 nM (data not shown), indicating the 50 nM level used for the biofilm experiments is within a relevant concentration range. Examination with CLSM showed that the AIPI treated biofilm sloughed off the flow cell over a period of 1-2 days (FIG. 2B and Video S1), suggesting that AIP-I activated a detachment mechanism. To confirm that AIP-I caused detachment, the inventors counted viable S. aureus cells in the effluent media (FIG. 2C). The concentration of bacteria in the effluent increased markedly 24-36 hours after AIP-I addition. In contrast, the number of bacteria in the biofilm effluent without AIP-I addition remained relatively constant. Computational analysis of the detachment phenotype indicated that 91.364.3% of the biomass dispersed within 48 hrs of AIP-I addition.

AIP-mediated biofilm detachment is a general phenomenon. Among S. aureus strains, there are four types of agr quorum-sensing systems. Each of these agr systems, referred to as agr-I through agr-IV, recognizes a unique AIP structure (AIP-I through AIP-IV). Through an intriguing mechanism of chemical communication, these varying quorum-sensing systems can be subdivided into three cross-inhibitory groups: agr-I/IV, agr-II, and agr-III. The activating signals of each group cross-inhibits the alternative signal receptors with surprising potency, a phenomenon termed “bacterial interference” (Ji et al., 1997). Since AIP-I and AIP-IV differ by only one amino acid and function interchangeably (Jarraud et al., 2000), they are grouped together in the classification scheme, although this assignment has been controversial (Goerke et al., 2003; McDowell et al., 2001).

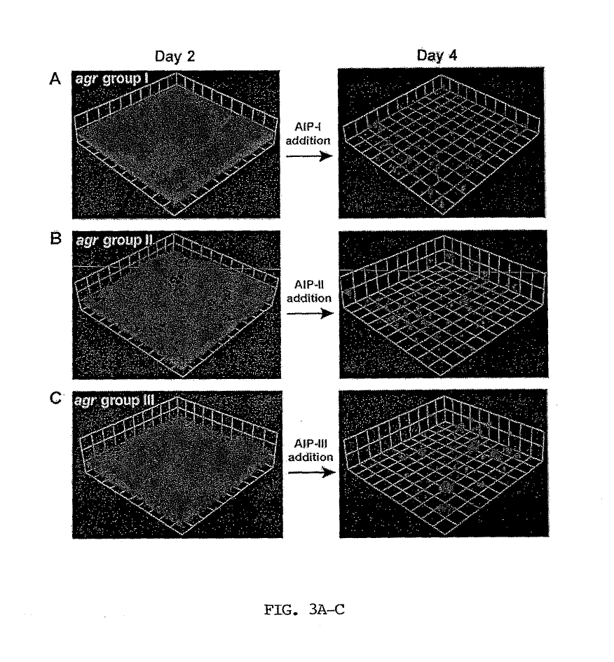

To determine the generality of the detachment mechanism, the inventors examined the effect of AIP addition using S. aureus strains representing different agr groups. The strains tested were (i) FRI1169, agr-I, toxic shock syndrome isolate (Sloane et al., 1991); (ii) SA502a (ATCC27217), nasal isolate and prototype agr-II strain (Ji et al., 1997; Shinefield et al., 1963); and (iii) ATCC25923, clinical agr-III isolate (Fux et al., 2004). When the correct AIP signal was added to 2-day old biofilms of each strain (FRI1169, AIP-1; SA502a, AIP-II; ATCC25923, AIP-III), signal addition resulted in robust detachment of each biofilm over a period of 48 hours (FIG. 3). These findings indicate biofilm detachment is a general S. aureus phenomenon that occurs in laboratory strains and clinical isolates, and functions across diverse agr systems.

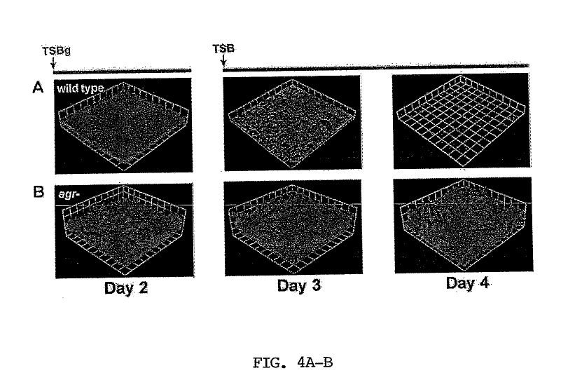

The timing and requirement of the agr system in detachment. If AIP was promoting biofilm detachment via the agr system, the inventors predicted that agr expression would be induced prior to detachment and an agr deficient mutant would not detach in response to AIP. To determine whether the agr system is activated prior to biofilm detachment, a dual fluorescent-labeled SH1000 strain was constructed with a constitutive RFP (PsarA-RFP, pAH9) and an agr responsive GFP reporter (PagrP3-GFP, pDB59). After two days of biofilm growth, the inventors added AIP-I to the biofilm flow medium and this resulted in strong induction of the GFP reporter (FIG. 4A), indicating activation of the agr system. As shown, the GFP reporter was clearly activated before dispersal of the biofilm cells. By the fourth day, all cells with detectable GFP expression detached from the biofilm. These observations provide convincing evidence that AIP activates the agr system prior to biofilm dispersal.

To further investigate the role of the agr system, the inventors utilized a mutant strain with a complete deletion of the agr locus (SH1001). Unlike the wild-type strain (FIG. 4A), the agr mutant biofilm harboring the same dual reporters did not respond to AIP-I treatment, as evidenced by a lack of GFP induction, and the mutant biofilm did not disperse (FIG. 4B). Similarly, addition of an inhibitory AIP (50 nM AIP-II) to the dual-labeled SH1000 biofilm failed to induce GFP expression, and again, the biofilm did not disperse (FIG. 4C). Taken together, these data demonstrate that an active agr quorum sensing system is necessary for AIP-mediated biofilm dispersal.

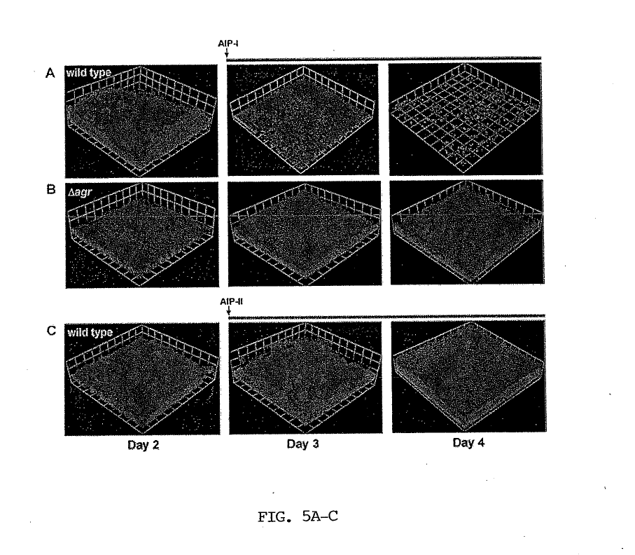

Changing environmental conditions can induce detachment. The inventors have demonstrated that low agr activity is important for biofilm formation and that activation of the agr system in established biofilms induces detachment. Considering changes to the physiochemical environment may occur in vivo, the inventors investigated whether an alteration in nutrient availability could reproduce the detachment phenotype. Again, two day flow cell biofilms were prepared with the dual-labeled strain (AH596) in TSBg (FIG. 5A). The glucose was removed and significant activation of the P3 promoter was apparent by monitoring GFP levels using CLSM (FIG. 5A), supporting the inventors' previous result (FIG. 1A). Once the agr system was activated, robust detachment from the flow cell was observed and monitored with CLSM (FIG. 5A). An agr deletion mutant did not respond to glucose depletion (FIG. 5B), indicating the detachment phenotype was dependent upon a functional agr system. These findings demonstrated that glucose depletion can disperse an S. aureus biofilm and again the detachment occurred through an agr-dependent mechanism. These experimental observations mirrored those with AIP addition and further support the apparent inverse correlation between agr activity and biofilm formation.

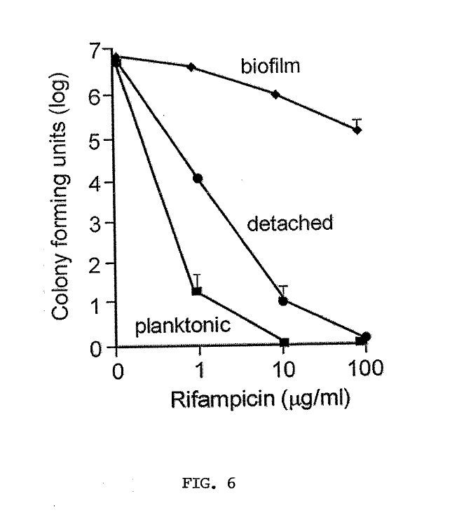

Detached S. aureus cells regain antibiotic sensitivity. Biofilm growth of S. aureus increases resistance to antimicrobials when compared to the planktonic growth mode (Fux et al., 2004; Yarwood et al., 2004). This Biofilm-mediated resistance hinders treatment of many chronic S. aureus biofilm related infections, including endocarditis, osteomyelitis, and indwelling medical device infections (Parsek and Singh, 2003; Costerton et al., 2003). Therefore, the inventors asked whether AIP-dispersed bacteria regained sensitivity to a clinically relevant antibiotic, rifampicin. To test this, the inventors collected detached cells from an AIP-treated biofilm effluent and compared resistance to intact biofilms exposed to different levels of rifampicin. Similar to previous antibiotic susceptibility results (Yarwood et al., 2004), even at the highest concentration tested (100 mg/ml), the level of rifampicin killing was about 2-log units of the biofilm biomass (FIG. 6). In contrast, the viability of detached cells displayed a different antibiotic response. At 10 mg/ml rifampicin, a 6-log decrease of viable cells was detected, and at 100 mg/ml, complete killing of the detached cells was observed (FIG. 6). The AIP-detached cells were more resistant than broth culture to comparable levels of rifampicin, suggesting parts of the detached biofilm may remain in emboli that are known to possess elevated antibiotic resistance (Fux et al., 2004). These observations demonstrated that S. aureus cells detached from a biofilm regain susceptibility to a clinical antibiotic.

The role of PIA in biofilm detachment. S. aureus possesses the ica-RADBC locus that is required to synthesize and generate an exopolysaccharide, which is referred to as the polysaccharide intracellular adhesin or PIA (also called PNAG). S. aureus is known to form biofilms through both ica-dependent and ica-independent mechanisms (O'Gara, 2007; Toledo-Arana et al., 2005). To gain insight on the biofilm detachment mechanism, the inventors sought to distinguish whether their S. aureus biofilms were dependent on PIA. In strain SH1000, the inventors constructed an Δica::Tet deletion mutant (strain AH595) using generalized transduction and confirmed the mutation with PCR and sequencing. In microtiter biofilm assays, they were unable to identify a biofilm phenotype (FIGS. 7A-B). Similarly in flow cell biofilms, they did not observe a defect in the ability of strain AH595 to form a biofilm (FIG. 7C). No difference was observed compared to flow cell biofilms of SH1000 grown in parallel (data not shown). While SH1000 is a derivative of 8325-4, and there are reports that the ica locus is required for 8325-4 derived strains to make a biofilm (Cramton et al., 1999), the ica locus was not required for biofilm formation under the present experimental conditions. Similar to the inventors' observations, an ica mutant of the clinical S. aureus isolate UAMS-1 displays no defect in microtiter and flow cell biofilm assays (Beenken et al., 2004). In contrast, when proteinase K was added to SH1000, biofilms were unable to develop in the microtiter plate format (data not shown), indicating the biofilms are forming through an ica-independent mechanism. These findings suggest that PIA is unlikely to have a role in biofilm detachment in the SH1000 strain background.

Investigating the biofilm detachment mechanism. Knowing the agr system is essential for biofilm detachment, what agr regulated products are responsible for the dispersal phenotype? In S. aureus strains that produce ica-independent biofilms, proteinase K eliminates adherence and biofilm formation (Toledo-Arana et al., 2005; Chaignon et al., 2007; O'Neill et al., 2007; Rohde et al., 2007), perhaps through cleavage of surface structures. S. aureus is coated with cell wall attached proteins that mediate adherence to a variety of substrates (Clarke and Foster, 2006), and some of these adhesins, such as biofilm-associated protein (BAP) and SasG are important for biofilm formation (Corrigan et al., 2007; Trotonda et al., 2005). It is also known that some surface adhesins, such as protein A and fibronectin-binding protein, are cleaved by the native S. aureus secreted proteases (Karlsson et al., 2001; McGavin et al., 1997). Considering the agr system regulates the secreted proteases (Dunman et al., 2001; Ziebandt et al., 2004), the inventors hypothesized that increased expression of extracellular proteases could be responsible for biofilm detachment.

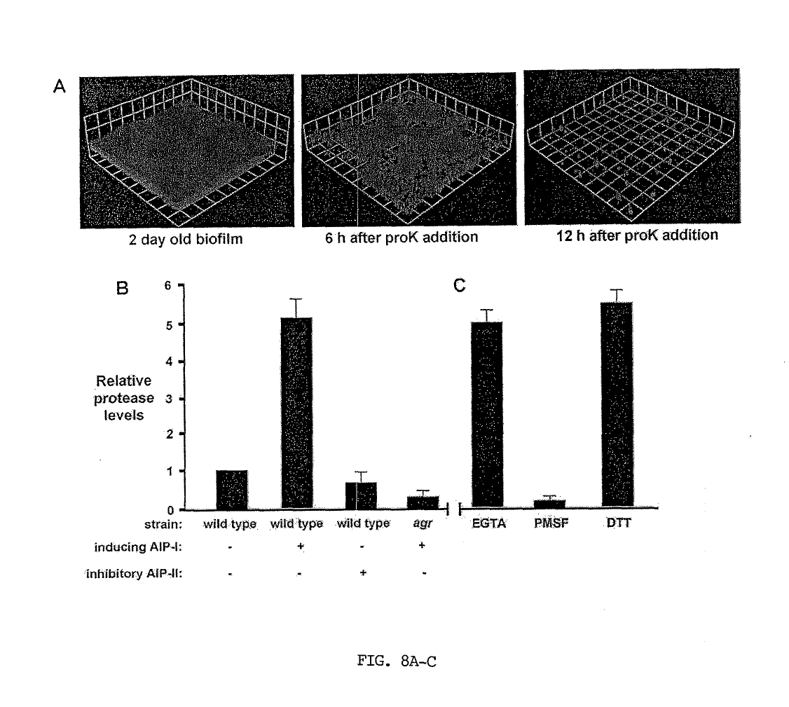

If S. aureus proteases have a role in detachment, proteinase K should be able to disperse an established biofilm. To test this proposal, proteinase K (2 mg/mL) was added to a SH1000 biofilm and resulted in rapid detachment over 12 hrs (FIG. 8A). With this preliminary observation, the inventors measured the levels of protease activity in effluents from biofilms with and without AIP-I addition using Azocoll (azo dye-impregnated collagen) reagent. As shown in FIG. 8B, the inventors detected a baseline level of protease activity in biofilm effluents without AIP-I addition and referenced other measurements to this baseline. With the addition of activating AIP-I, the protease activity increased approximately five-fold compared to a biofilm with no AIP-I treatment. As anticipated, addition of inhibitory AIP-II reduced the level of proteolytic activity in the effluent. Similarly, an agr mutant biofilm supplemented with activating AIP-I displayed very low levels of extracellular proteases (FIG. 8B).

There are 10 known extracellular proteases produced by most S. aureus strains and expression of all these enzymes is controlled by the agr system (Novick, 2003; Dunman et al., 2001; Ziebandt et al., 2004). These 10 proteases include the metalloprotease aureolysin (aur), two cysteine proteases (scpA and sspB), and seven serine proteases (sspA (V8) and sp1ABCDEF) (Dubin, 2002). To elucidate what class(es) of proteases are prevalent in AIP-treated biofilms, the effluent from a detaching biofilm was assayed for protease activity in the presence of protease inhibitors or activating agents. The addition of EGTA, an inhibitor of the metalloprotease aureolysin (Kavanaugh et al., 2007), had a negligible effect on overall protease activity (FIG. 8C). The addition of PMSF, a potent serine protease inhibitor, however reduced overall protease activity to almost undetectable levels. Lastly, the addition of DTT, a reducing agent used to activate thiol proteases (Fournier et al., 2001), did not significantly change protease activity in the effluents. These results suggest that serine proteases are the dominant, detectable secreted protease in AIP-treated biofilms.

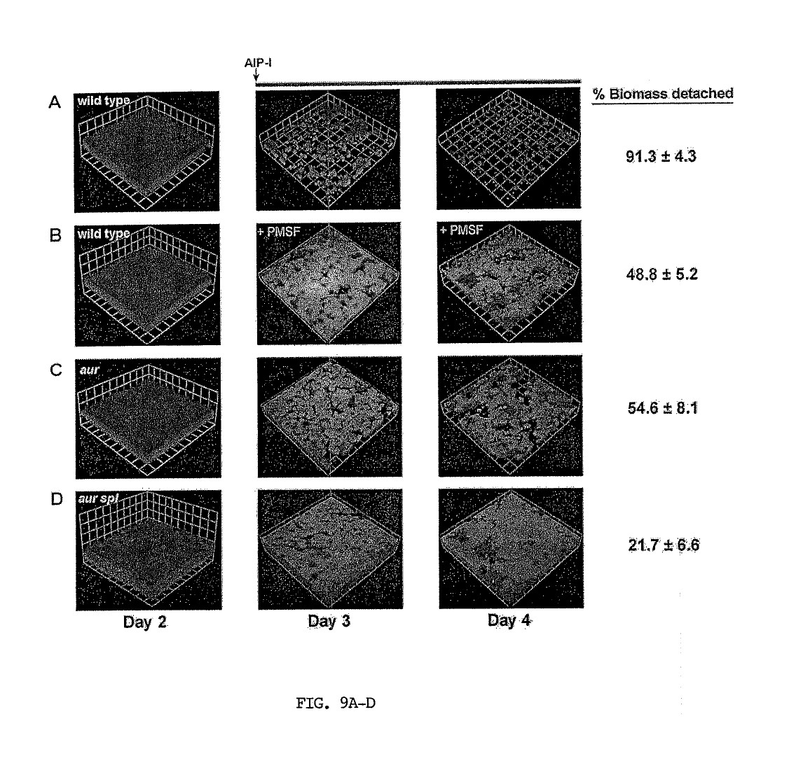

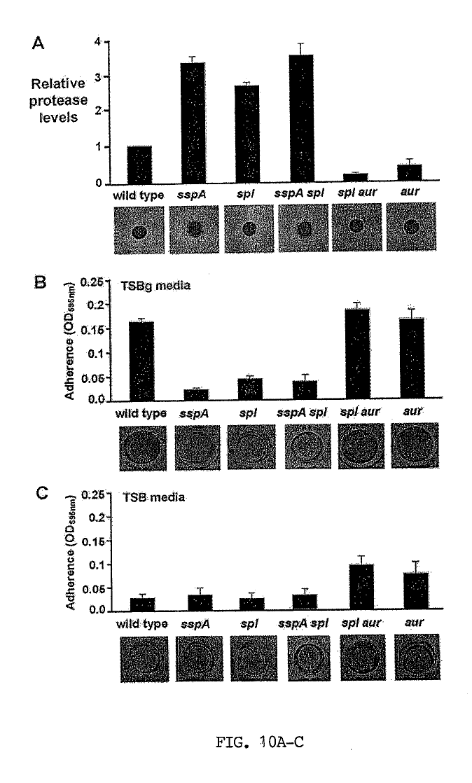

Protease activity is required for biofilm detachment. With the observation that serine proteases are abundant in detaching biofilms, the inventors examined the effect of a serine protease inhibitor on AIP-mediated detachment. The addition of 10 mM PMSF in combination with AIP-I to an S. aureus biofilm significantly reduced the level of detachment compared with AIP-I alone (FIGS. 9A vs. 9B). However, 48.8% (65.2) of the biomass still detached indicating that serine proteases are necessary but not sufficient for complete detachment. To further examine the mechanism, knock-out mutations were constructed in the genes encoding the V8 (SspA) and Sp1ABCDEF serine proteases. Surprisingly, sspA::Tet and Dsp1::Erm single mutants, and an sspA::Tet Dsp1::Erm double mutant, all increased extracellular protease levels (FIG. 10A) and eliminated biofilm formation under microtiter plate conditions (FIGS. 10B-C).

To block other extracellular proteases, a mutation was constructed in the gene encoding aureolysin (Aur). Aur is a metalloprotease that is required to initiate a zymogen activation cascade (Shaw et al., 2004; Rice et al., 2001), starting with the V8 protease (Drapeau, 1978), which in turn activates the SspB cysteine protease (Massimi et al., 2002). The activation mechanism of the ScpA cysteine protease remains unresolved (Shaw et al., 2004). In contrast to the serine protease mutations, introduction of the Δaur deletion into S. aureus reduced extracellular protease levels (FIG. 10A) and did not affect biofilm formation (FIG. 10B). Interestingly, under conditions of high agr activity, the Δaur deletion displayed improved biofilm formation versus wild-type (FIG. 10C). In biofilm detachment tests, the Daur mutant reduced AIP-mediated detachment, but 54.6% (68.1) of the biomass still detached (FIG. 9C). Considering the Sp1 proteases are not zymogens (Popowicz et al., 2006), the inventors examined the combined effects of the Aur cascade and the Sp1 proteases by constructing an Δaur Dsp1::Erm double mutant. The Δaur Dsp1 strain possessed very low levels of extracellular protease activity (FIG. 10A) and had a minor enhancement in biofilm formation (FIG. 10B). Similar to the Daur mutant, the Δaur Dsp1 double mutant also displayed improved biofilm formation versus wild-type under conditions of high agr activity (FIG. 10C). After AIP-I addition, only 21.7% (66.6) of the Δaur Dsp1 mutant biomass detached in comparison to 91.3 (64.3) of the wild-type strain (FIG. 9D). These experiments indicate that the extracellular proteases have anti-biofilm properties and they demonstrate that agr-mediated biofilm detachment requires the activity of these proteases.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,415,732
U.S. Pat. No. 4,458,066,
U.S. Pat. No. 4,946,778
U.S. Pat. No. 5,354,855
U.S. Pat. No. 5,795,715
U.S. Pat. No. 5,888,773
U.S. Pat. No. 5,889,136
U.S. Pat. No. 6,337,385
U.S. Pat. No. 6,953,833
U.S. Patent Publication 2007/0185016
Allison et al., FEMS Microbiol. Lett., 167:179-184, 1998.
Applegate and Bryers, Biotechnol. Bioeng., 37:15-25, 1991.
Atkinson et al., Mol. Microbiol., 33:1267-1277, 1999.
Banin et al., Appl. Environ. Microbiol., 72:2064-2069, 2006.
Barraud et al., J. Bacteriol., 188:7344-7353, 2006.
Bashore et al., Curr. Probl. Cardiol., 31:274-352, 2006.
Beenken et al., Infect. Immun., 71:4206-4211, 2003.
Beenken et al., J. Bacteriol., 186:4665-4684, 2004.
Bischoff et al., J. Bacteriol., 186:4085-4099, 2004.
Biswas et al., FEMS Microbiol. Lett., 259:260-268, 2006.
Blevins et al., Infect. Immun., 70:470-480, 2002.
Boles et al., Mol. Microbiol., 57:1210-1223, 2005.
Bosher and Labouesse, Nat. Cell. Biol., 2(2):E31-E36, 2000.
Caiazza and O'Toole, J. Bacteriol., 185:3214-3217, 2003.
Capaldi et al., Biochem. Biophys. Res. Comm., 74(2):425-433, 1977.
Caplen et al., Gene, 252(1-2):95-105, 2000.
Chaignon et al., Appl. Microbiol. Biotechnol., 75:125-132, 2007.
Cheung et al., Infect. Immun., 67:1331-1337, 1999.
Clarke and Foster, Adv. Microb. Physiol., 51:187-224, 2006.
Cook et al., Cell, 27:487-496, 1981.
Corrigan et al., Microbiology, 153:2435-2446, 2007.
Costerton et al., J. Clin. Invest., 112:1466-1477, 2003.
Costerton, Clin. Orthop. Relat. Res., 7-11, 2005.
Cramton et al., Infect. Immun., 67:5427-5433, 1999.
Cucarella et al., J. Bacteriol., 183:2888-2896, 2001.
Davey and O'Toole, Microbiol. Mol. Biol. Rev., 64:847-867, 2000.
Davey et al., J. Bacteriol., 185:1027-1036, 2003.
Davies et al., Appl. Environ. Microbiol., 59:1181-1186, 1993.
Davies et al., Science, 280:295-298, 1998.
Davies, Nat. Rev. Drug Discov., 2:114-122, 2003.
Davis et al., J. Biol. Chem., 252:6544-6553, 1977.
Dow et al., Proc. Natl. Acad. Sci. USA, 100:10995-11000, 2003.
Drapeau, J. Bacteriol., 136:607-613, 1978.
Dubin, Biol. Chem., 383:1075-1086, 2002.
Dunman et al., J. Bacteriol., 183:7341-7353, 2001.
Elbashir et al., Nature, 411(6836):494-498, 2001.
Elbashir et al., EMBO, 20(23):6877-6888, 2001.
Elbashir et al., Genes Dev., 5(2):188-200, 2001.
Entenza et al., Infect. Immun., 73(2):990-998, 2005.
Fire et al., Nature, 391(6669):806-811, 1998.
Fleming et al., J. Bacteriol., 188:7686-7688, 2006.
Forster and Symons, Cell, 48:211-220, 1987.
Fournier and Klier, Mol. Microbiol., 41:247-261, 2001.
Fowler et al., Jama, 293:3012-3021, 2005.
Furukawa et al., J. Bacteriol., 188:1211-1217, 2006.
Fux et al., J. Bacteriol., 186:4486-4491, 2004.
Gerlach et al., Nature (London), 328:802-805, 1987.
Gertz et al., Mol. Gen. Genet., 261:558-566, 1999.
Goerke et al., J. Infect. Dis., 188:250-256, 2003.
Gotz et al., Chem. Phys. Lipids, 93:15-25, 1998.
Grishok et al., Science, 287:2494-2497, 2000.
Hall-Stoodley and Stoodley, Trends Microbiol., 13:7-10, 2005.
Harris and Richards, Injury, 37(Suppl 2):S3-14, 2006.
Heilmann et al., Mol. Microbiol., 24:1013-1024, 1997.
Heydorn et al., Microbiology, 146(Pt 10):2395-2407, 2000.
Horsburgh et al., J. Bacteriol., 184:5457-5467, 2002.
Huber et al., Microbiology, 147:2517-2528, 2001.
Hunt et al., Appl. Environ. MicrobioL, 70:7418-7425, 2004.
Irie et al., FEMS Microbiol. Lett., 250:237-243, 2005.
Izano et al., Appl. Environ. Microbiol., 74:470-476, 2008.
Jarraud et al., Infect. Immun., 70:631-641, 2002.
Jarraud et al., J. Bacteriol., 182:6517-6522, 2000.
Ji et al., Science, 276:2027-2030, 1997.
Joly et al., Antimicrob. Agents Chemother., 31:1632-1639, 1987.
Joyce, Nature, 338:217-244, 1989.
Kaplan et al., J. Bacteriol., 185:4693-4698, 2003.
Kaplan et al., J. Bacteriol., 186:8213-8220, 2004.
Karlsson et al., Infect. Immun., 69:4742-4748, 2001.
Kavanaugh et al., Mol. Microbiol., 65:780-798, 2007.
Ketting et al., Cell, 99(2):133-141, 1999.
Kim and Cook, Proc. Natl. Acad. Sci. USA, 84(24):8788-8792, 1987.
Knobloch et al., Infect. Immun., 72:3838-3848, 2004.
Knobloch et al., J. Bacteriol., 183:2624-2633, 2001.
Kong et al., Int. J. Med. Microbiol., 296:133-139, 2006.
Kullik and Giachino, Arch. Microbiol., 167:151-159, 1997.
Leid et al., Infect. Immun., 70:6339-6345, 2002.
Lin et al., J. Gen. Virol., 80:91-96, 1999.
Lyon et al., Biochemistry, 41:10095-10104, 2002.
Malone et al., Appl. Environ. Microbiol., 73(9):6036-6044, 2007.
Maria-Litran et al., Infect. Immun., 70:4433-4440, 2002.
Massimi et al., J. Biol. Chem., 277:41770-41777, 2002.
Mayville et al., Proc. Natl. Acad. Sci. USA, 96:1218-1223, 1999.
McDowell et al., Mol. Microbiol., 41:503-512, 2001.
McGavin et al., Infect. Immun., 65:2621-2628, 1997.
Michel and Westhof, J. Mol. Biol., 216:585-610, 1990.
Montgomery et al., Proc. Natl. Acad. Sci. USA, 95:15502-15507, 1998.
Morgan et al., J. Bacteriol., 188:7335-7343, 2006.
Musk et al., Chem. Biol., 12:789-796, 2005.
Novick, Methods Enzymol., 204:587-636, 1991.
Novick, Mol. Microbiol., 48:1429-1449, 2003.
O'Gara, FEMS Microbiol. Lett., 270:179-188, 2007.
O'Neill et al., J. Clin. Microbiol., 45:1379-1388, 2007.
Oshida et al., Proc. Natl. Acad. Sci. USA, 92:285-289, 1995.
Pane-Farre et al., Int. J. Med. Microbiol., 296:237-258, 2006.
Parsek and Singh, Annu. Rev. Microbiol., 57:677-701, 2003.
Parsek and Singh, Annu. Rev. Microbiol., 57:677-701, 2003.
Patel, Clin. Orthop. Relat. Res., 41-47, 2005.
PCT Appln. PCT US2007/087663
PCT Appln. WO 00/44914
PCT Appln. WO 01/68836
PCT Appln. WO 84/03564
PCT Appln. WO 99/32619
PCT Appln. WO 01/36646
Popowicz et al., J. Mol. Biol., 358: 270-279, 2006.
Puskas et al., J. Bacteriol., 179:7530-7537, 1997.
Qiu et al., J. Biol. Chem., 280:16695-16704, 2005.
Rachid et al., J. Bacteriol., 192:6824-6826, 2000.
Reed et al., Infect. Immun., 69:1521-1527, 2001.
Regassa et al., Infect. Immun., 60:3381-3388, 1992.
Reinhold-Hurek and Shub, Nature, 357:173-176, 1992.
Rice et al., Infect. Immun., 69:159-169, 2001.
Rice et al., J. Bacteriol., 187:3477-3485, 2005.
Rice et al., Proc. Natl. Acad. Sci. USA, 104:8113-8118, 2007.
Rohde et al., Biomaterials, 28:1711-1720, 2007.
Rupp et al., Appl. Environ. Microbiol., 71:2175-2178, 2005.
Sarver et al., Science, 247:1222-1225, 1990.
Sauer et al., J. Bacteriol., 186:7312-7326, 2004.
Scanlon et al., Proc. Natl. Acad. Sci. USA, 88:10591-10595, 1991.
Schenk and Laddaga, FEMS Microbiol. Lett., 73:133-138, 1992.
Shaner et al., Nat. Biotechnol., 22:1567-1572, 2004.
Shanks et al., Infect. Immun., 73:4596-4606, 2005.
Sharp and Zamore, Science, 287:2431-2433, 2000.
Sharp, Genes Dev., 13:139-141, 1999.
Shaw et al., Microbiology, 150:217-228, 2004.
Shinefield et al., Am. J. Dis. Child., 105:646-654, 1963.
Sloane et al., FEMS Microbiol. Lett., 62:239-244, 1991.
Suciu et al., Mol. Microbiol., 21:181-195, 1996.
Sung et al., Infect. Immun., 74:2947-2956, 2006.
Tabara et al., Cell, 99(2):123-132, 1999.
Thormann et al., J. Bacteriol., 187:1014-1021, 2005.
Thormann et al., J. Bacteriol., 188:2681-2691, 2006.
Toledo-Arana et al., J. Bacteriol., 187:5318-5329, 2005.
Trotonda et al., J. Bacteriol., 187:5790-5798, 2005.
Valle et al., Mol. Microbiol., 48:1075-1087, 2003.
Vuong et al., J. Infect. Dis., 182:1688-1693, 2000.
Weinrick et al., J. Bacteriol., 186:8407-8423, 2004.
Whitchurch et al., Science, 295:1487, 2002.
Wincott et al., Nucleic Acids Res., 23(14):2677-2684, 1995.
Wright et al., Proc. Natl. Acad. Sci. USA, 101:16168-161-73, 2004.
Wright et al., Proc. Natl. Acad. Sci. USA, 102:1691-1696, 2005.
Yarwood et al., J. Bacteriol., 186:1838-1850, 2004.
Zhang and Ji, J. Bacteriol., 186:6706-6713, 2004.
Zhang and Pierson, Appl. Environ. Microbiol., 67:4305-4315, 2001.
Ziebandt et al., Proteomics, 1:480-493, 2001.
Ziebandt et al., Proteomics, 4:3034-3047, 2004.

1. A method of inhibiting a bacterial biofilm comprising contacting a biofilm-forming bacterium with an activator of an agr quorum-sensing system.

2. The method of claim 1, wherein said agr quorum-sensing system is agr-I.

3. The method of claim 1, wherein said agr quorum-sensing system is agr-II.

4. The method of claim 1, wherein said agr quorum-sensing system is agr-III.

5. The method of claim 1, wherein said agr quorum-sensing system is agr-IV.

6. The method of claim 1, wherein said activator is an auto-inducing peptide (AIP).

7. The method of claim 1, wherein said bacterium is Staphylococcus aureus or Psuedomonas aeruginosa.

8. The method of claim 1, further comprising contacting said bacterium with an antibiotic or antiseptic agent.

9. The method of claim 1, wherein inhibiting comprises inhibiting biofilm formation.

10. The method of claim 1, wherein inhibiting comprises inhibiting biofilm growth.

11. The method of claim 1, wherein inhibiting comprises reducing biofilm size.

12. The method of claim 1, wherein inhibiting comprises promoting detachment of bacteria from a formed biofilm.

13. The method of claim 1, wherein said biofilm or biofilm-forming bacterium is located in a subject.

14. The method of claim 13, wherein said subject is a mammalian subject.

15. The method of claim 14, wherein said mammalian subject is a human subject.

16. The method of claim 13, wherein said subject comprises an in-dwelling medical device.

17. The method of claim 16, wherein said in-dwelling medical device is a catheter, a pump, endotracheal tube, a nephrostomy tube, a stent, an orthopedic device, a suture, a or prosthetic valve.

18. The method of claim 16, wherein said catheter is a vascular catheter, an urinary catheter, a peritoneal catheter, an epidural catheter, a central nervous system catheter, central venous catheter, an arterial line catheter, a pulmonary artery catheter, or a peripheral venous catheter.

19. The method of claim 13, wherein said biofilm or biofilm-forming bacterium is located on a wound dressing.

20. The method of claim 13, wherein said biofilm or biofilm-forming bacterium is located on a tissue surface.

21. The method of claim 20, wherein said tissue surface is a heart valve, bone or epithelia.

22. The method of claim 1, wherein said biofilm or biofilm-forming bacterium is located on an inanimate surface.

23. The method of claim 19, wherein said inanimate surface is a floor, a table-top, a counter-top, a medical device surface, a wheelchair surface, a bed surface, a sink, a toilet, a filter, a valve, a coupling, or a tank.

24. The method of claim 1, wherein said biofilm is located in an industrial system.

25. The method of claim 24, wherein said industrial system is a heating/cooling system, a water provision or purification system, or a medical pump system.

26. The method of claim 16, further comprising coating said in-dwelling medical device with said inhibitor prior to implantation.

27. The method of claim 1, wherein said inhibitor is a SigB inhibitor.

28. A method of preventing biofilm formation secondary to nosocomial infection in a subject comprising administering to said subject an activator of an agr quorum-sensing system in combination with an antibiotic

29. The method of claim 28, wherein said nosocomial infection is pneumonia, bacteremia, a urinary tract infection, a catheter-exit site infection, and a surgical wound infection.

30. A method of restoring antibiotic sensitivity to a bacterium located in a biofilm comprising contacting said bacterium with an activator of an agr quorum-sensing system.

31. The method of claim 30, further comprising administerting to said subject an antibiotic.


SEQUENCE LISTING (4 sequences)

SEQ ID NO: 1 - protein, 8 aa, Synechocystis PCC6803
YSTCDFIM

SEQ ID NO: 2 - protein, 9 aa, Synechocystis PCC6803
GVNACSSLF

SEQ ID NO: 3 - protein, 7 aa, Synechocystis PCC6803
INCDFLL

SEQ ID NO: 4 - protein, 8 aa, Synechocystis PCC6803
YSTCYFIM